(19) United States
(12) Patent Application Publication
(10) Pub. No.: US 2020/0270697 A1
(43) Pub. Date: Aug. 27, 2020

(54) METHOD FOR PREDICTING ORGAN TRANSPLANT REJECTION USING NEXT-GENERATION SEQUENCING

(71) Applicant: EONE DIAGNOMICS GENOME CENTER CO., LTD., Incheon (KR)

(72) Inventors: Min Seob LEE, Incheon (KR); Sun Jae KWON, Incheon (KR)

(21) Appl. No.: 16/833,587

(22) Filed: Mar. 28, 2020

Related U.S. Application Data
(63) Continuation-in-part of application No. 15/566,484, filed on Oct. 13, 2017.

(30) Foreign Application Priority Data
Apr. 14, 2015 (KR) 10-2015-0052649

(51) Int. Cl. C12Q 1/6883; C12Q 1/6869; G16B 30/00; G16H 10/40; G16H 50/20

(52) U.S. Cl. CPC C12Q 1/6883; C12Q 1/6869; G16B 30/00; G01N 2800/50; G16H 50/20; G01N 2800/245; G16H 10/40

(57) ABSTRACT

The present invention demonstrates a non-invasive method for organ transplant rejection prediction by measuring the ratio between donor-specific nucleic acid sequences and recipient-specific nucleic acid sequences in a biological sample obtained from an organ transplant recipient. More precisely, it relates to a method that comprises of analyzing a biological sample (e.g., blood) obtained from an organ transplant recipient to measure the ratio between donor-derived marker sequences and recipient-derived marker sequences, having three or more markers selected from the markers listed in Tables 1 to 10, and thereby predicting organ transplant rejection based on the ratio. The method of predicting organ transplant rejection using next-generation sequencing (NGS) or digital base amplification based on the present invention can be applied even to a minute amount of a sample. This method is rapid, inexpensive, enables rapid data analysis, is applicable irrespective of the organ type and races in the world, and also it can detect the probability of sequencing error. Thus, the method of the present invention is useful for non-invasive prediction of organ transplant rejection.

### CROSS-REFERENCE TO RELATED APPLICATIONS

This is a continuation-in-part under the provisions of 35 U.S.C. § 120 of U.S. patent application Ser. No. 15/566,484 filed Oct. 13, 2017 as a U.S. national phase under the provisions of 35 U.S.C. § 371 of International Patent Application No. PCT/KR2015/005905 filed Jun. 11, 2015, which in turn claims priority of Korean Patent Application No. 10-2015-0052649 filed Apr. 14, 2015. The disclosures of U.S. patent application Ser. No. 15/566,484, International Patent Application No. PCT/KR2015/005905, and Korean Patent Application No. 10-2015-0052649 are hereby incorporated herein by reference in their respective entireties, for all purposes.

### TECHNICAL FIELD

The present invention relates to a method of non-invasively predicting organ transplant rejection by measuring the ratio between donor-specific nucleic acid sequences and recipient-specific nucleic acid sequences in a biological sample obtained from an organ transplant recipient, and more particularly to a method of predicting organ transplant rejection based on the results of measuring the ratio between donor-derived marker sequences and recipient-derived marker sequences by analyzing a biological sample (e.g., blood) obtained from an organ transplant recipient.

### BACKGROUND ART

Accurate and timely diagnosis of organ transplant rejection in an organ transplant recipient is essential for survival of the organ transplant recipient. However, methods for diagnosing organ transplant rejection, which are currently used, have many disadvantages. For example, gold standard for diagnosing heart transplant rejection is examining tissue at each time point with surgery for heart biopsy, however, this methods shows many problems including high costs, variability between tissue biopsy physicians, and severe patient discomfort (F. Saraiva et al., Transplant. Proc. Vol. 43, pp. 1908-1912, 2011).

In order to overcome such limitations, non-invasive methods have been used, such as a method for measuring gene expression signals which tend to increase when organ transplant rejection occurs, a method for measuring the level of immune proteins, and the like. However, these methods also pose limitations as they tend to produce high false positive results due to the complex cross-reactivity of various immune responses, and are based on tissue-specific gene expression signals.

In the late 1990s, cell-free donor-derived DNA (cfdDNA) was detected in the urine and blood of organ transplant recipients (J. Zhang et al., Clin. CHem. Vol. 45, pp. 1741-1746, 1999., Y. M. Lo et al., Lancet, Vol. 351, pp. 1329-1330, 1998). Based on this finding, methods for non-invasive diagnosis of organ transplant rejection have been proposed. For example, donor-specific DNA in a female recipient of organ from a male donor can be analyzed using various molecular and chemical assays that detect Y chromosome (T. K. Sigdel et al., Transplantation, Vol. 96, pp. 97-101, 2013). However, the cfdDNA is present in minute quantity, whereas the background DNA is present in abundance. Thus, a highly specific and sensitive method for analyzing this cfdDNA is required.

Next-generation sequencing (NGS) has the capacity of overcoming such limitations and is becoming more and more popular. The next-generation sequencing technique can produce huge amount of data within a short span of time, unlike the existing methods. Thus, this technique is both time and cost effective for individual genome sequencing. The next-generation sequencing technique also provides an unprecedented opportunity to detect disease-causing genes in Mendelian diseases, rare diseases, cancers and the like. Extraordinary progress has been made on genome sequencing platforms and the sequencing data analysis costs have gradually reduced. In the next-generation sequencing technique, DNA is extracted from a sample and mechanically fragmented, followed by size-specific library construction which is used for sequencing. Initial sequencing data are produced while repeating the association and dissociation of four complementary nucleotides with one base unit by using high-throughput sequencing system. Subsequently, bioinformatics-based analysis steps are performed which includes initial data trimming, mapping, genetic mutation identification, and mutation annotation. The analysis leads to the identification of genetic mutations that may affect diseases and various biological phenotypes. Thus, the next-generation sequencing technique contributes to the creation of new added values through the development and commercialization of new therapeutic agents. The next-generation sequencing technique can not only be used for DNA analysis, but also for RNA and methylation analysis. This includes whole-exome sequencing (WES) that captures and sequences only protein-encoding exome regions. This whole-exome sequencing technique is a method that produce sequences of the region encoding a protein having the most direct connection with the development of disease. This technique is widely used, because sequencing of only the exome region is more cost-effective as compared to sequencing of the whole genome. The modification of the whole-exome sequencing technique is popularly known as targeted sequencing. This sequencing technique has the capacity of detecting genetic mutation in the region of interest by using a designed probe. The probe captures only the genetic region of interest, which in turn is used for the detection of genetic mutation in the major oncogene of interest. This technique is relatively easy to perform and can be achieved by significantly lower costs. This sequencing technique is referred to as targeted sequencing.

It is a well-known fact that the use of this next-generation sequencing technique makes it possible to analyze all nucleic acids present in a sample, and thus is highly useful for the analysis of cfdDNA that is present in a desired sample at a very low concentration. For example, Iwijin De Vlaminck et al. performed the analysis of 565 samples obtained from 65 heart transplant patients over time which indicated that the level of cfdDNA in the samples from the recipients were elevated when organ transplant rejection appeared (Iwijin De Vlaminck et al., Sci. Transl. Med. Vol. 6, 241ra77, 2014).

However, this method has limitations; it requires considerable amount of time and cost as it analyzes whole genome data.

Accordingly, the present inventors have made extensive efforts to solve the above-described problems, and as a result, have found that, when markers shown in Table 1 to 10 or 15 below are amplified to a size of less than 200 bp and used in next-generation sequencing, cfDNA in a sample can be used intact and, at the same time, analysis sensitivity and accuracy are maintained and analysis time and cost are significantly decreased, thereby completing the present invention.

### DISCLOSURE OF INVENTION

### Technical Problem

It is an object of the present invention to provide a method of predicting organ transplant rejection in a biological sample, obtained from a recipient who received an organ from a donor, by next-generation sequencing (NGS) or digital base amplification

Another object of the present invention is to provide a computer system comprising a computer readable medium encoded with a plurality of instructions for controlling a computing system to perform an operation of predicting organ transplant rejection in a biological sample, obtained from a recipient who received an organ from a donor, by use of next-generation sequencing (NGS) or digital base amplification.

### Technical Solution

To achieve the above object, the present invention provides a method of predicting organ transplant rejection in a biological sample, obtained from a recipient who received an organ from a donor, by next-generation sequencing (NGS) or digital base amplification, the method comprising the steps of:

non-invasively obtaining a biological sample, which contains donor-derived and recipient-derived cell-free nucleic acid molecules, from a recipient who received an organ from a donor;

amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in cell-free nucleic acid molecules isolated from the biological sample;

analyzing the amplified sequences by next-generation sequencing (NGS) or digital base amplification;

based on the analysis of the sequences, determining the ratio between each of the donor-derived marker sequences and each of the recipient-derived marker sequences; and

comparing the ratio with one or more cutoff values.

The present invention also provides a method of predicting organ transplant rejection in a biological sample, obtained from a recipient who received an organ from a donor, by next-generation sequencing (NGS) or digital base amplification, the method comprising the steps of:

non-invasively obtaining a biological sample, which contains donor-derived and recipient-derived cell-free nucleic acid molecules, from a recipient who received an organ from a donor;

amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in cell-free nucleic acid molecules isolated from the biological sample; analyzing the amplified sequences by next-generation sequencing (NGS) or digital base amplification;

based on the analysis of the sequences, determining the ratio between each of the donor-derived marker sequences and each of the recipient-derived marker sequences; and

measuring the ratio over time, and predicting whether the recipient will have transplant rejection, graft dysfunction or organ failure when the ratio each of the donor-derived marker sequences increases.

The present invention also provides a computer system comprising a computer readable medium encoded with a plurality of instructions for controlling a computing system to perform an operation of predicting organ transplant rejection in a biological sample, obtained from a recipient who received an organ from a donor, by use of next-generation sequencing (NGS) or digital base amplification,

wherein the biological sample contains donor-derived and recipient-derived cell-free nucleic acid molecules from a recipient who received an organ from a donor, and

wherein the operation comprises the steps of:

receiving data obtained by analyzing rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in the cell-free nucleic acid molecules isolated from the biological sample, by use of next-generation sequencing (NGS) or digital base amplification; based on the analysis of the sequences, determining the ratio between each of the donor-derived marker sequences and each of the recipient-derived marker sequences;

comparing the ratio with one or more cutoff values; and

based on the comparison, predicting whether or not organ transplant rejection in the recipient will be present.

Set out below are marker sequences and corresponding primers of SEQ ID NOs: 1-652.

| | Forward Primer SEQ ID NO:, |
|---|---|
| Marker Sequence | Reverse Primer SEQ ID NO: |
| rs6490946 | 1, 2 |
| rs6490683 | 15, 16 |
| rs9552733 | 29, 30 |
| rs4770463 | 43, 44 |
| rs4770601 | 57, 58 |
| rs1886969 | 71, 72 |
| rs4771157 | 85, 86 |
| rs1927830 | 99, 100 |
| rs169332 | 113, 114 |
| rs524566 | 127, 128 |
| rs2764618 | 141, 142 |
| rs1832265 | 155, 156 |
| rs6562599 | 169, 170 |
| rs9544845 | 183, 184 |
| rs7236653 | 197, 198 |
| rs1284419 | 211, 212 |
| rs892430 | 225, 226 |
| rs17533 | 239, 240 |
| rs1786648 | 253, 254 |
| rs2419027 | 267, 268 |
| rs9949868 | 281, 282 |
| rs2090036 | 295, 296 |
| rs1893455 | 309, 310 |
| rs1593579 | 323, 324 |
| rs2222370 | 337, 338 |
| rs4798479 | 351, 352 |
| rs16951664 | 365, 366 |
| rs12606001 | 379, 380 |
| rs2821796 | 393, 394 |
| rs375484 | 407, 408 |
| rs2242661 | 421, 422 |
| rs4817371 | 435, 436 |
| rs2256417 | 449, 450 |
| rs413285 | 463, 464 |
| rs1014604 | 477, 478 |
| rs1102617 | 491, 492 |
| rs2183557 | 505, 506 |
| rs863075 | 519, 520 |
| rs2825608 | 533, 534 |
| rs12482714 | 547, 548 |
| rs2826390 | 561, 562 |
| rs2774494 | 3, 4 |
| rs7334546 | 17, 18 |
| rs9510597 | 31, 32 |
| rs9317882 | 45, 46 |
| rs943049 | 59, 60 |
| rs306395 | 73, 74 |
| rs1231048 | 87, 88 |
| rs9508327 | 101, 102 |
| rs9508716 | 115, 116 |
| rs798963 | 129, 130 |
| rs6561924 | 143, 144 |
| rs9564535 | 157, 158 |
| rs9572308 | 171, 172 |
| rs9574740 | 185, 186 |
| rs8095071 | 199, 200 |
| rs9303869 | 213, 214 |
| rs12327010 | 227, 228 |
| rs273696 | 241, 242 |
| rs8096542 | 255, 256 |
| rs2419041 | 269, 270 |
| rs8097023 | 283, 284 |
| rs8097433 | 297, 298 |
| rs9947829 | 311, 312 |
| rs4133291 | 325, 326 |
| rs2320747 | 339, 340 |
| rs10502290 | 353, 354 |
| rs602212 | 367, 368 |
| rs2027667 | 381, 382 |
| rs8134986 | 395, 396 |
| rs377191 | 409, 410 |
| rs2822661 | 423, 424 |
| rs1018676 | 437, 438 |
| rs2245411 | 451, 452 |
| rs2205533 | 465, 466 |
| rs2223079 | 479, 480 |
| rs8127266 | 493, 494 |
| rs1573442 | 507, 508 |
| rs1826318 | 521, 522 |
| rs2825610 | 535, 536 |
| rs2826117 | 549, 550 |
| rs2826392 | 563, 564 |
| rs9285110 | 5, 6 |
| rs6490713 | 19, 20 |
| rs9552874 | 33, 34 |
| rs9553022 | 47, 48 |
| rs9581121 | 61, 62 |
| rs9551233 | 75, 76 |
| rs9551406 | 89, 90 |
| rs2892463 | 103, 104 |
| rs7327256 | 117, 118 |
| rs2188584 | 131, 132 |
| rs9570290 | 145, 146 |
| rs1508494 | 159, 160 |
| rs7990298 | 173, 174 |
| rs7988209 | 187, 188 |
| rs1787013 | 201, 202 |
| rs2487257 | 215, 216 |
| rs716510 | 229, 230 |
| rs273701 | 243, 244 |
| rs4800786 | 257, 258 |
| rs2729429 | 271, 272 |
| rs11081004 | 285, 286 |
| rs1390431 | 299, 300 |
| rs8090831 | 313, 314 |
| rs9945284 | 327, 328 |
| rs4798412 | 341, 342 |
| rs2298583 | 355, 356 |
| rs8092926 | 369, 370 |
| rs329027 | 383, 384 |
| rs12482146 | 397, 398 |
| rs38 6838 | 411, 412 |
| rs11088040 | 425, 426 |
| rs926130 | 439, 440 |
| rs2736084 | 453, 454 |
| rs2823795 | 467, 468 |
| rs9978408 | 481, 482 |
| rs208932 | 495, 496 |
| rs2824762 | 509, 510 |
| rs7278137 | 523, 524 |
| rs128365 | 537, 538 |
| rs977660 | 551, 552 |
| rs2826396 | 565, 566 |
| rs876165 | 7, 8 |
| rs9506919 | 21, 22 |
| rs9805694 | 35, 36 |
| rs7992072 | 49, 50 |
| rs9553390 | 63, 64 |
| rs9512046 | 77, 78 |
| rs1778797 | 91, 92 |
| rs1570614 | 105, 106 |
| rs7986681 | 119, 120 |
| rs9534174 | 133, 134 |
| rs11839815 | 147, 148 |
| rs1876583 | 161, 162 |
| rs9542852 | 175, 176 |
| rs9575364 | 189, 190 |
| rs9952107 | 203, 204 |
| rs786018 | 217, 218 |
| rs6508351 | 231, 232 |
| rs9303900 | 245, 246 |
| rs16943649 | 259, 260 |
| rs17711702 | 273, 274 |
| rs878971 | 287, 288 |
| rs12967616 | 301, 302 |
| rs9967142 | 315, 316 |
| rs9967440 | 329, 330 |
| rs6506332 | 343, 344 |
| rs3786355 | 357, 358 |
| rs11660737 | 371, 372 |
| rs403093 | 385, 386 |
| rs431864 | 399, 400 |
| rs4588273 | 413, 414 |
| rs760345 | 427, 428 |
| rs2822965 | 441, 442 |
| rs975336 | 455, 456 |
| rs2823809 | 469, 470 |
| rs2226798 | 483, 484 |
| rs211962 | 497, 498 |
| rs8132870 | 511, 512 |
| rs2825560 | 525, 526 |
| rs2825688 | 539, 540 |
| rs1786427 | 553, 554 |
| rs2096905 | 567, 568 |
| rs4770032 | 9, 10 |
| rs9510334 | 23, 24 |
| rs2148443 | 37, 38 |
| rs736081 | 51, 52 |
| rs9551135 | 65, 66 |
| rs9507631 | 79, 80 |
| rs9508080 | 93, 94 |
| rs514669 | 107, 108 |
| rs914532 | 121, 122 |
| rs9534262 | 135, 136 |
| rs10507416 | 149, 150 |
| rs9564626 | 163, 164 |
| rs12586094 | 177, 178 |
| rs7323558 | 191, 192 |
| rs7231366 | 205, 206 |
| rs1030133 | 219, 220 |
| rs7228099 | 233, 234 |
| rs1785745 | 247, 248 |
| rs939139 | 261, 262 |
| rs9957591 | 275, 276 |
| rs1662304 | 289, 290 |
| rs12966492 | 303, 304 |
| rs1567612 | 317, 318 |
| rs6506138 | 331, 332 |
| rs694182 | 345, 346 |
| rs12185460 | 359, 360 |
| rs4239334 | 373, 374 |
| rs8087127 | 387, 388 |
| rs435081 | 401, 402 |
| rs2013669 | 415, 416 |
| rs467140 | 429, 430 |
| rs2822973 | 443, 444 |
| rs2823145 | 457, 458 |
| rs2823983 | 471, 472 |
| rs2824238 | 485, 486 |
| rs2824376 | 499, 500 |
| rs2211938 | 513, 514 |
| rs2825576 | 527, 528 |
| rs1474092 | 541, 542 |
| rs2826259 | 555, 556 |
| rs13433508 | 569, 570 |
| rs9509249 | 11, 12 |
| rs9510340 | 25, 26 |
| rs7323182 | 39, 40 |
| rs1830926 | 53, 54 |
| rs4770771 | 67, 68 |
| rs9512063 | 81, 82 |
| rs4573787 | 95, 96 |
| rs10478 | 109, 110 |
| rs7339250 | 123, 124 |
| rs4942486 | 137, 138 |
| rsl2876644 | 151, 152 |
| rs9542105 | 165, 166 |
| rs9543171 | 179, 180 |
| rs7325529 | 193, 194 |
| rs7237577 | 207, 208 |
| rs968906 | 221, 222 |
| rs12961741 | 235, 236 |
| rs1785739 | 249, 250 |
| rs6508502 | 263, 264 |
| rs1026123 | 277, 278 |
| rs11081037 | 291, 292 |
| rs6507196 | 305, 306 |
| rs1145560 | 319, 320 |
| rs11872146 | 333, 334 |
| rs341237 | 347, 348 |
| rs16951141 | 361, 362 |
| rs8095514 | 375, 376 |
| rs1023868 | 389, 390 |
| rs439146 | 403, 404 |
| rs2822618 | 417, 418 |
| rs461853 | 431, 432 |
| rs2822975 | 445, 446 |
| rs2823152 | 459, 460 |
| rs2824133 | 473, 474 |
| rs13046156 | 487, 488 |
| rs243594 | 501, 502 |
| rs2825516 | 515, 516 |
| rs2825578 | 529, 530 |
| rs2825824 | 543, 544 |
| rs1786388 | 557, 558 |
| rs12483578 | 571, 572 |
| rs9509441 | 13, 14 |
| rs1834547 | 27, 28 |
| rs9510775 | 41, 42 |
| rs4770597 | 55, 56 |
| rs9507577 | 69, 70 |
| rs2497654 | 83, 84 |
| rs9579214 | 97, 98 |
| rs9506275 | 111, 112 |
| rs4943119 | 125, 126 |
| rs7330025 | 139, 140 |
| rs1361029 | 153, 154 |
| rs9592665 | 167, 168 |
| rs2031446 | 181, 182 |
| rs4796869 | 195, 196 |
| rs11080646 | 209, 210 |
| rs10210 | 223, 224 |
| rs1834545 | 237, 238 |
| rs8096605 | 251, 252 |
| rs11083386 | 265, 266 |
| rs336279 | 279, 280 |
| rs1790649 | 293, 294 |
| rs4799910 | 307, 308 |
| rs991045 | 321, 322 |
| rs4407150 | 335, 336 |
| rs9958938 | 349, 350 |
| rs7234111 | 363, 364 |
| rs7235989 | 377, 378 |
| rs2775537 | 391, 392 |
| rs416083 | 405, 406 |
| rs2822648 | 419, 420 |
| rs2822809 | 433, 434 |
| rs1002460 | 447, 448 |
| rs2823169 | 461, 462 |
| rs10470201 | 475, 476 |
| rs9984531 | 489, 490 |
| rs243601 | 503, 504 |
| rs1491658 | 517, 518 |
| rs2825583 | 531, 532 |
| rs377685 | 545, 546 |
| rs8128523 | 559, 560 |
| rs9786291 | 645, 646 |
| rs9786140 | 647, 648 |
| rs11096453 | 649, 650 |
| rs9786194 | 651, 652 |
| rs2058276 | 631, 632 |
| rs1358368 | 633, 634 |
| rs9786773 | 635, 636 |
| rs9786885 | 637, 638 |
| rs2571764 | 639, 640 |
| rs3096835 | 641, 642 |
| rs1864469 | 643, 644 |
| rs9786101 | 617, 618 |
| rs3991109 | 619, 620 |
| rs9785716 | 621, 622 |
| rs7067226 | 623, 624 |
| rs7067297 | 625, 626 |
| rs9785897 | 627, 628 |
| rs1276034 | 629, 630 |
| rs17307670 | 603, 604 |
| rs9786876 | 605, 606 |
| rs9785659 | 607, 608 |
| rs9786121 | 609, 610 |
| rs9786111 | 611, 612 |
| rs9786276 | 613, 614 |
| rs2161775 | 615, 616 |
| rs13304168 | 589, 590 |
| rs16980558 | 591, 592 |
| rs16980586 | 593, 594 |
| rs16980588 | 595, 596 |
| rs9786824 | 597, 598 |
| rs765557 | 599, 600 |
| rs9786386 | 601, 602 |
| rs9983568 | 575, 576 |
| rs8129332 | 577, 578 |
| rs229441 | 579, 580 |
| rs2826506 | 581, 582 |
| rs2014509 | 583, 584 |
| rs11096435 | 585, 586 |
| rs13304202 | 587, 588 |
| rs2826399 | 573, 574 |

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a conceptual view depicting a method for the prediction of organ transplant rejection by next-generation sequencing (NGS). As shown in FIG. 1, a biological sample (e.g., blood) is collected non-invasively from a patient, and circulating cell-free DNA is isolated from the biological sample. A selected marker set (shown in table 15) in the sample is amplified by multiplex PCR and analyzed by short read length next generation sequencing to count the ratio between marker alleles, thereby predicting organ transplant rejection.

FIG. 2 shows an illustrative marker sequence rs149098 (shown with SNP “R” at position 81, corresponding to “n” in the corresponding SEQ ID NO: 653) and forward and reverse primers of SEQ ID NOs: 654 and 655. FIG. 2 illustrates that when a single marker is amplified using a designed primer and analyzed, NGS can be very quickly performed because a target SNP site (shown as “R” in FIG. 2, and represented as “n” in SEQ ID NO: 653, which may be any one of “A”, “T”, “C”, or “G”) is located immediately following the primer.

FIG. 3 depicts the results obtained by mixing DNAs to artificially make organ transplantation conditions for 2023 markers selected from markers shown in Table 1 to 10, and measuring the percentages of donor-derived SNP markers in a transplant recipient.

FIG. 4 depicts the results of measuring each SNP marker in a sample comprising artificially mixed DNAs.

FIG. 5 depicts the result of measuring fluctuations of ddcfDNA in respective samples by the time.

### ADVANTAGEOUS EFFECTS

The method of prediction of organ transplant rejection by next-generation sequencing (NGS) or digital base amplification according to the present invention is applicable even for minute amount of sample. This method is rapid, inexpensive, enables rapid data analysis, and is applicable irrespective of the types of organs and races in the world, Also it can detect the probability of the sequencing error. Thus, the method of the present invention is vital for non-invasive prediction of organ transplant rejection.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well-known and commonly employed in the art.

As used herein, the term “next-generation sequencing (NGS)” means a technique in which the whole genome is fragmented and the fragments are sequenced in a high-throughput manner. The term includes the technologies of Agilent, Illumina, Roche and Life Technologies. In a broad sense, the term includes third-generation sequencing technologies such as Pacificbio, Nanopore Technology and the like, and also the fourth-generation sequencing technologies.

As used herein, the term “organ transplant rejection” includes both acute and chronic transplant rejections. “Acute transplant rejection (AR)” occurs when the donor's organ is considered exogenous by the recipient's immune system. “Acute transplant rejection” implies that the recipient's immune cells penetrate a transplanted organ, resulting in destruction of the transplant organ. Acute transplant rejection occurs very rapidly, and it generally occurs within weeks after organ transplantation surgery. Generally, acute transplant rejection can be inhibited or suppressed by immunosuppressants such as rampamycin, cyclosprin A, anti-CD4 monoclonal antibody and the like. “Chronic transplant rejection (CR)” generally occurs within several months or years after organ transplantation. Organ fibrosis that occurs in all kinds of chronic transplant rejection is a common phenomenon that reduces the function of each organ. For example, chronic transplant rejection in a transplanted lung occurs leads to fibrotic reaction which destroys the airways leading to pneumonia (bronchiolitis obliterans). Furthermore, when chronic transplant rejection occurs in a transplanted heart, it will result in fibrotic atherosclerosis. Similarly, the chronic transplant rejection in a transplanted kidney leads to obstructive nephropathy, nephrosclerosis, tubulointerstitial nephropathy or the like. Chronic transplant rejection also results in ischemic insult, denervation of a transplanted organ, hyperlipidemia and hypertension symptoms associated with immunosuppressants. As used herein, the term “biological sample” refers to any sample that is obtained from a recipient and contains one or more nucleic acid molecule(s) of interest.

The term “nucleic acid” or “polynucleotide” refers to a deoxyribonucleic acid (DNA) or ribonucleic acid (RNA) and a polymer thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al, MoI. Cell. Probes 8:91-98 (1994)). The term nucleic acid is used interchangeably with gene, cDNA, mRNA, small noncoding RNA, micro RNA (miRNA), Piwi-interacting RNA, and short hairpin RNA (shRNA) encoded by a gene or locus.

As used herein, the term “single nucleotide polymorphism (SNP)” refers to a single nucleotide difference between a plurality of individuals within a single species. For example, when an rs7988514 marker present in chromosome 13 of a transplant donor is C/C and the marker in a transplant recipient is T/T, the method of the present invention can be used to analyze the ratio of the donor-derived marker in the blood of the recipient to thereby predict organ transplant rejection.

The term “cutoff value” as used herein means a numerical value whose value is used to arbitrate between two or more states (e.g. normal state and organ transplant rejection state) of classification for a biological sample. For example, if the ratio of a donor-derived marker in the blood of a recipient is greater than the cutoff value, the recipient is classified as being in the organ transplant rejection state; or if the ratio of the donor-derived marker in the blood of the recipient is less than the cutoff value, the recipient is classified as being in the normal state.

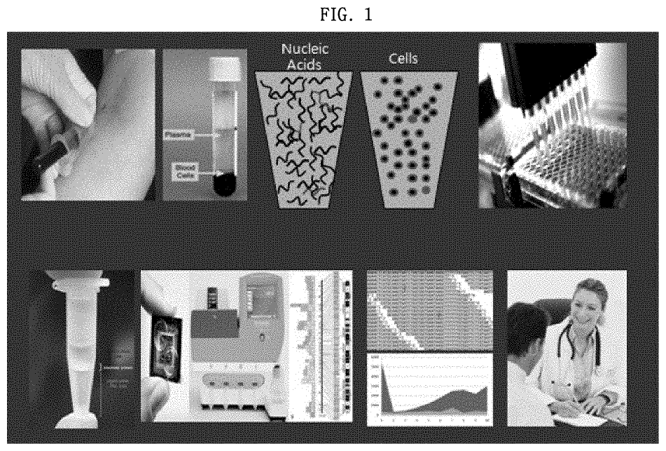

In the present invention, it was found that when a biological sample collected from a recipient is analyzed by short read length next-generation sequencing or digital base amplification by using at least three markers selected from the list of markers shown in Tables 1 to 10 or 15 (FIG. 1), organ transplant rejection in the biological sample can be quickly predicted with high accuracy.

Therefore, in one aspect, the present invention is directed to a method of predicting organ transplant rejection in a biological sample, obtained from a recipient who received an organ from a donor, by next-generation sequencing (NGS) or digital base amplification, the method comprising the steps of:

non-invasively obtaining a biological sample, which contains donor-derived and recipient-derived cell-free nucleic acid molecules, from a recipient who received an organ from a donor;

amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in cell-free nucleic acid molecules isolated from the biological sample; analyzing the amplified sequences by next-generation sequencing (NGS) or digital base amplification;

based on the analysis of the sequences, determining the ratio between each of the donor-derived marker sequences and each of the recipient-derived marker sequences; and

comparing the ratio with one or more cutoff values.

In the present invention, the marker sequences listed in Tables 1 to 10 or 15 can be used as single nucleotide polymorphism (SNP) markers which are bi-allelic, are in agreement with the a Hardy-Weinberg distribution and have a minor allele frequency of 0.4 or greater.

In the present invention, the marker numbers (rs numbers) listed in Tables 1 to 10 or 15 may have reference SNP numbers that can be searched in dbSNP database (http://www.ncbi.nlm.nih.gov/snp) of NCBI.

TABLE 1
Markers used in the present invention

| Marker | Marker | Marker | Marker | Marker | Marker | Marker |
|---|---|---|---|---|---|---|
| number | number | number | number | number | number | number |
| rs1000160 | rs1483303 | rs2296122 | rs3773445 | rs6565831 | rs899968 | rs9978408 |
| rs1000501 | rs1491658 | rs2296348 | rs377685 | rs6565969 | rs904654 | rs9979609 |
| rs1002460 | rs1495816 | rs2297256 | rs378108 | rs6565990 | rs906629 | rs9980589 |
| rs1003092 | rs1495965 | rs2297291 | rs3786203 | rs6566067 | rs912441 | rs9980734 |
| rs10035179 | rs1498553 | rs2298437 | rs3786355 | rs6566186 | rs912697 | rs9980852 |
| rs10048391 | rs1501230 | rs2298583 | rs3787732 | rs6566286 | rs914163 | rs9980934 |
| rs10048862 | rs1501233 | rs2318993 | rs3788190 | rs6566554 | rs914231 | rs9981016 |
| rs10049125 | rs1501871 | rs2320747 | rs3788200 | rs6566669 | rs914232 | rs9982310 |
| rs10057967 | rs1506008 | rs232374 | rs378872 | rs6566675 | rs914532 | rs9982473 |
| rs1006757 | rs1508494 | rs232381 | rs3795494 | rs6566862 | rs915800 | rs9983057 |
| rs10069510 | rs1511151 | rs2328975 | rs379605 | rs6567221 | rs915876 | rs9983351 |
| rs1007300 | rs1512473 | rs2329327 | rs3802981 | rs6579927 | rs918823 | rs9983568 |
| rs10074004 | rs1518036 | rs2330396 | rs3803196 | rs659897 | rs924895 | rs9984531 |
| rs10075717 | rs1519126 | rs2330572 | rs3805015 | rs660207 | rs926130 | rs9985011 |
| rs10078065 | rs1526589 | rs2332023 | rs3806 | rs660236 | rs928299 | rs9985019 |
| rs10083274 | rs1530330 | rs2332026 | rs3809346 | rs660622 | rs9285110 | rs9985057 |
| rs10085762 | rs153119 | rs2332240 | rs3810590 | rs660811 | rs9285254 | rs999104 |
| rs1009823 | rs153283 | rs233616 | rs3817 | rs661100 | rs9285297 | |
| rs10098835 | rs1532846 | rs233621 | rs3819177 | rs661293 | rs9292170 | |
| rs1010392 | rs1533434 | rs2337483 | rs3826616 | rs662792 | rs9300377 | |
| rs1010559 | rs1535904 | rs234787 | rs3844038 | rs6650458 | rs9300518 | |
| rs1013059 | rs1536780 | rs235310 | rs384901 | rs6650723 | rs9300569 | |
| rs10140137 | rs1536807 | rs235329 | rs3850193 | rs665479 | rs9300647 | |
| rs1014209 | rs1542578 | rs236043 | rs3853682 | rs6662560 | rs9300921 | |
| rs1014604 | rs1545310 | rs2362839 | rs385501 | rs6678950 | rs9301149 | |
| rs1015820 | rs1549060 | rs2366188 | rs3856791 | rs6680365 | rs9301441 | |
| rs10158288 | rs1553108 | rs2388919 | rs3864997 | rs671441 | rs9301695 | |
| rs10164030 | rs1553295 | rs2390878 | rs3865418 | rs673220 | rs930189 | |
| rs1018676 | rs1554936 | rs2390998 | rs3865419 | rs674929 | rs9303869 | |
| rs10210 | rs1556817 | rs239340 | rs3866900 | rs6762432 | rs9303900 | |
| rs10222177 | rs1560669 | rs2395891 | rs386838 | rs6769917 | rs9304336 | |

TABLE 2
Markers used in the present invention

| Marker | Marker | Marker | Marker | Marker | Marker | Marker |
|---|---|---|---|---|---|---|
| number | number | number | number | number | number | number |
| rs1058396 | rs1682914 | rs2571764 | rs4268850 | rs703505 | rs946228 | rs10915584 |
| rs10742709 | rs1690551 | rs2575177 | rs428424 | rs7062 | rs946231 | rs10935501 |
| rs10744938 | rs169332 | rs2576036 | rs4284535 | rs706466 | rs949741 | rs1757889 |
| rs1075906 | rs16943649 | rs2576038 | rs4284740 | rs7067226 | rs949931 | rs17591231 |
| rs1077550 | rs16950864 | rs2581732 | rs429133 | rs7067297 | rs950408 | rs277665 |
| rs10780042 | rs16951141 | rs2585481 | rs430043 | rs7099777 | rs9506275 | rs2779134 |
| rs10781417 | rs16951664 | rs2585495 | rs4306614 | rs7139787 | rs9506919 | rs445593 |
| rs10790400 | rs16978368 | rs2586776 | rs431864 | rs7139997 | rs9507577 | rs4456612 |
| rs1079139 | rs16980558 | rs2586778 | rs4319623 | rs7140005 | rs9507631 | rs7234111 |
| rs1079174 | rs16980586 | rs2586779 | rs432294 | rs714831 | rs950772 | rs7234383 |
| rs10799636 | rs16980588 | rs2587428 | rs4329028 | rs716117 | rs9508080 | rs9521192 |
| rs10840837 | rs17041964 | rs25876 | rs4346468 | rs716510 | rs9508327 | rs952134 |
| rs10851201 | rs17069898 | rs2591518 | rs4346469 | rs7186326 | rs9508716 | rs10903035 |
| rs10853392 | rs17070149 | rs2628125 | rs4349043 | rs7206898 | rs9509249 | rs10915311 |
| rs10853603 | rs17071467 | rs2641114 | rs4349054 | rs721247 | rs9509441 | rs1754514 |
| rs10854400 | rs17080696 | rs2641962 | rs435081 | rs7226953 | rs9509516 | rs17550441 |
| rs10856953 | rs17084208 | rs2687899 | rs4372773 | rs7226979 | rs9510334 | rs2776341 |
| rs10858469 | rs170962 | rs269286 | rs4375553 | rs7227268 | rs9510340 | rs2776344 |
| rs10866988 | rs17184424 | rs2699323 | rs4380323 | rs7228099 | rs9510597 | rs4452046 |
| rs10869149 | rs1720839 | rs271374 | rs438064 | rs7228812 | rs9510775 | rs4454841 |
| rs10869157 | rs17232531 | rs271397 | rs4383238 | rs7229278 | rs9512046 | rs7233802 |
| rs10870724 | rs17248234 | rs2729429 | rs4384683 | rs7229644 | rs9512063 | rs7233985 |
| rs10870932 | rs1725235 | rs2736084 | rs4389803 | rs7229967 | rs9514560 | rs9520400 |
| rs10871180 | rs17307670 | rs273696 | rs439146 | rs722998 | rs9514663 | rs9521146 |
| rs10871550 | rs17326281 | rs273701 | rs4398676 | rs7230288 | rs9515124 | |
| rs10871620 | rs1734848 | rs2747740 | rs4402665 | rs7230661 | rs9515621 | |
| rs10871641 | rs17351137 | rs275948 | rs4402842 | rs7230860 | rs9515774 | |
| rs10871815 | rs17363863 | rs2762171 | rs4407150 | rs7231029 | rs9516644 | |
| rs10875612 | rs174047 | rs2764618 | rs4409964 | rs7231046 | rs9516904 | |
| rs10880836 | rs1748124 | rs2765327 | rs4428160 | rs7231112 | rs9518743 | |
| rs10889256 | rs17513940 | rs2774494 | rs4430618 | rs7231366 | rs9518903 | |
| rs10889523 | rs17518254 | rs2775138 | rs4440160 | rs7232672 | rs9518972 | |
| rs10899035 | rs17533 | rs2775537 | rs444435 | rs7233515 | rs9520132 | |

TABLE 3
Markers used in the present invention

| Marker | Marker | Marker | Marker | Marker | Marker | Marker |
|---|---|---|---|---|---|---|
| number | number | number | number | number | number | number |
| rs10937406 | rs17591266 | rs2780746 | rs446716 | rs7234990 | rs9521472 | rs11151454 |
| rs10937408 | rs17686332 | rs2782462 | rs4468699 | rs7235005 | rs9521488 | rs11151514 |
| rs10942130 | rs17711702 | rs2783084 | rs448247 | rs7235160 | rs9521801 | rs1790428 |
| rs10955093 | rs1772587 | rs2786712 | rs448503 | rs7235654 | rs9521832 | rs1790584 |
| rs10955174 | rs17740268 | rs2793734 | rs4495668 | rs7235891 | rs9521853 | rs2825610 |
| rs10955420 | rs1774918 | rs2793736 | rs4497518 | rs7235930 | rs9522262 | rs2825688 |
| rs10972552 | rs17754863 | rs2794243 | rs4508511 | rs7235989 | rs9523648 | rs466448 |
| rs1102617 | rs17765723 | rs2794247 | rs4510132 | rs7236090 | rs9524400 | rs467140 |
| rs1105513 | rs17781378 | rs2803220 | rs451826 | rs7236427 | rs9525095 | rs7277441 |
| rs1105856 | rs1778797 | rs2803348 | rs4520729 | rs7236653 | rs9525149 | rs7277926 |
| rs11069237 | rs17794801 | rs2807441 | rs4522508 | rs7237517 | rs9525158 | rs9532436 |
| rs11071215 | rs1779843 | rs2821796 | rs4525375 | rs7237577 | rs9525300 | rs9533146 |
| rs11080646 | rs17800754 | rs2822618 | rs4539677 | rs7237747 | rs9525641 | rs11151426 |
| rs11081004 | rs17804894 | rs2822648 | rs4544336 | rs7237774 | rs9525643 | rs11151452 |
| rs11081037 | rs1783099 | rs2822661 | rs455508 | rs7239234 | rs9526222 | rs1788648 |
| rs11081555 | rs1783305 | rs2822809 | rs455921 | rs723940 | rs9526312 | rs1788658 |
| rs11082705 | rs1783395 | rs2822965 | rs4573787 | rs7240004 | rs9526400 | rs2825583 |
| rs11083008 | rs1783404 | rs2822973 | rs4576968 | rs7240257 | rs9526792 | rs2825608 |
| rs11083386 | rs17836226 | rs2822975 | rs458029 | rs7240294 | rs9527084 | rs4661514 |
| rs1108522 | rs1785739 | rs2823145 | rs4583369 | rs7240363 | rs9527138 | rs466277 |
| rs11088040 | rs1785745 | rs2823152 | rs4588087 | rs7240404 | rs9527905 | rs7276176 |
| rs11088302 | rs1786388 | rs2823169 | rs4588273 | rs7240429 | rs9528696 | rs7277076 |
| rs11088405 | rs1786427 | rs2823795 | rs4611350 | rs7241051 | rs9528931 | rs9531615 |
| rs11088861 | rs1786648 | rs2823809 | rs4613170 | rs7241461 | rs9529287 | rs9532420 |
| rs11096435 | rs1787013 | rs2823983 | rs4617713 | rs7241510 | rs9529809 | |
| rs11096453 | rs1787186 | rs2824133 | rs461853 | rs7241718 | rs9529814 | |
| rs1111937 | rs1787292 | rs2824238 | rs4628 | rs7242966 | rs9530505 | |
| rs11135235 | rs1787301 | rs2824376 | rs4638449 | rs7243620 | rs9530604 | |
| rs1114342 | rs1787337 | rs2824762 | rs4650520 | rs7244347 | rs9530721 | |
| rs11148802 | rs1787435 | rs2825516 | rs4653036 | rs7245332 | rs9530981 | |
| rs11150946 | rs1787557 | rs2825560 | rs465353 | rs725040 | rs953109 | |
| rs11151009 | rs1787577 | rs2825576 | rs465446 | rs7260507 | rs9531243 | |
| rs11151180 | rs1788002 | rs2825578 | rs4661295 | rs7275842 | rs9531587 | |

TABLE 4
Markers used in the present invention

| Marker | Marker | Marker | Marker | Marker | Marker | Marker |
|---|---|---|---|---|---|---|
| number | number | number | number | number | number | number |
| rs11151657 | rs1790649 | rs2825824 | rs4677496 | rs7278004 | rs9533177 | rs11662474 |
| rs11151684 | rs1790875 | rs2826117 | rs4685212 | rs7278137 | rs9533397 | rs11662612 |
| rs11151698 | rs1792668 | rs2826259 | rs4686407 | rs7278676 | rs9533738 | rs1890306 |
| rs11151892 | rs1792674 | rs2826390 | rs4687889 | rs7279020 | rs9534174 | rs1891132 |
| rs11152060 | rs1792687 | rs2826392 | rs468837 | rs7279626 | rs9534262 | rs2828798 |
| rs11152170 | rs1806487 | rs2826395 | rs468849 | rs7280367 | rs9534330 | rs2828800 |
| rs11152242 | rs1807783 | rs2826396 | rs469303 | rs7280538 | rs9534515 | rs4799055 |
| rs11152264 | rs1808693 | rs2826399 | rs469353 | rs7280591 | rs9534596 | rs4799198 |
| rs11164166 | rs1810129 | rs2826506 | rs470490 | rs7280941 | rs9534638 | rs732569 |
| rs11167694 | rs1817141 | rs2826718 | rs4747351 | rs7281206 | rs9535880 | rs7326426 |
| rs11208377 | rs1819894 | rs2826721 | rs4750494 | rs7281674 | rs9536346 | rs9545554 |
| rs1125807 | rs1826318 | rs2826737 | rs4757240 | rs728174 | rs9536415 | rs9545559 |
| rs1143914 | rs1829651 | rs2826803 | rs4761518 | rs7281853 | rs9538268 | rs11661072 |
| rs1145560 | rs1830926 | rs2826807 | rs4770032 | rs7282582 | rs9538278 | rs11661849 |
| rs1146888 | rs1832265 | rs2826949 | rs4770463 | rs7282876 | rs9539175 | rs1888469 |
| rs1152991 | rs1833277 | rs2826959 | rs4770597 | rs7283077 | rs9539877 | rs1888514 |
| rs1153294 | rs1833304 | rs2827038 | rs4770601 | rs7283399 | rs9539893 | rs2828506 |
| rs1153295 | rs1833486 | rs2827433 | rs4770771 | rs729809 | rs9540071 | rs2828793 |
| rs1156026 | rs1834545 | rs2827527 | rs4771157 | rs7304820 | rs9540450 | rs4798412 |
| rs115750 | rs1834547 | rs2827528 | rs4771638 | rs7310809 | rs9540451 | rs4798479 |
| rs11595762 | rs1851043 | rs2827530 | rs4771695 | rs7317338 | rs9540627 | rs7325068 |
| rs11617291 | rs1854100 | rs2827874 | rs4771833 | rs7317341 | rs9540642 | rs7325529 |
| rs11617562 | rs1855259 | rs2827965 | rs4771904 | rs7317430 | rs9541479 | rs9545224 |
| rs11617606 | rs1864469 | rs2827987 | rs4772278 | rs7319926 | rs9541813 | rs9545244 |
| rs11618168 | rs1866337 | rs2828001 | rs4772857 | rs7319976 | rs9542105 | |
| rs11619265 | rs1866986 | rs2828023 | rs4772937 | rs7320145 | rs9542137 | |
| rs11619462 | rs1870592 | rs2828055 | rs4773212 | rs7321115 | rs9542383 | |
| rs11620473 | rs1874864 | rs2828061 | rs4773395 | rs7321584 | rs9542852 | |
| rs11659206 | rs1874921 | rs2828089 | rs4773402 | rs7322458 | rs9542951 | |
| rs11659463 | rs1876583 | rs2828151 | rs4773838 | rs7322868 | rs9542969 | |
| rs11659969 | rs188446 | rs2828155 | rs4784207 | rs7323182 | rs9543171 | |
| rs11660213 | rs1886969 | rs2828263 | rs4784376 | rs7323558 | rs9544749 | |
| rs11660737 | rs1887718 | rs2828500 | rs4796869 | rs7324970 | rs9544845 | |

TABLE 5
Markers used in the present invention

| Marker | Marker | Marker | Marker | Marker | Marker | Marker |
|---|---|---|---|---|---|---|
| number | number | number | number | number | number | number |
| rs11664190 | rs1891948 | rs2828802 | rs4799910 | rs7326820 | rs9545852 | rs12184876 |
| rs11664478 | rs189204 | rs2829066 | rs4800786 | rs7326944 | rs9545853 | rs12185460 |
| rs11664727 | rs1892681 | rs2829115 | rs4800967 | rs7327180 | rs9545861 | rs1970678 |
| rs11665106 | rs1893455 | rs2829214 | rs4800970 | rs7327256 | rs9545903 | rs1972415 |
| rs11665385 | rs1893654 | rs2829432 | rs4800973 | rs7327729 | rs9546633 | rs2833935 |
| rs11701849 | rs1893657 | rs2829445 | rs4816597 | rs7329520 | rs9546677 | rs2834208 |
| rs11701901 | rs1893673 | rs2829614 | rs4816610 | rs7330025 | rs9547087 | rs4886217 |
| rs11702340 | rs1895076 | rs2829674 | rs4816681 | rs7331003 | rs9547646 | rs4890312 |
| rs1176270 | rs1898165 | rs2829887 | rs4817097 | rs7331794 | rs9548869 | rs9561936 |
| rs1183856 | rs1904177 | rs2830048 | rs4817371 | rs7332180 | rs9548880 | rs9561953 |
| rs11839815 | rs1908593 | rs2830194 | rs4817609 | rs7333280 | rs9548930 | rs1217618 |
| rs11872146 | rs1910660 | rs2830424 | rs4817685 | rs7333503 | rs9549172 | rs1218307 |
| rs11872403 | rs191482 | rs2830437 | rs4817890 | rs7333648 | rs9549293 | rs195700 |
| rs11872509 | rs1920083 | rs2830604 | rs4817891 | rs733398 | rs9551135 | rs1970668 |
| rs11872828 | rs1923732 | rs2830643 | rs4818015 | rs7334111 | rs9551233 | rs2833846 |
| rs11873161 | rs1923771 | rs2830811 | rs4818108 | rs7334546 | rs9551406 | rs2833916 |
| rs11876001 | rs1923886 | rs2830841 | rs4818144 | rs7334805 | rs9552733 | rs4885878 |
| rs11876772 | rs1924417 | rs2830856 | rs4818160 | rs7335163 | rs9552874 | rs4885880 |
| rs11877050 | rs1925857 | rs2831057 | rs4818179 | rs7335426 | rs9553022 | rs735862 |
| rs11877617 | rs1926264 | rs2831350 | rs4818561 | rs7335836 | rs9553390 | rs736081 |
| rs11910048 | rs1926614 | rs2831378 | rs4819090 | rs7335944 | rs9554579 | rs9561532 |
| rs11910807 | rs1926616 | rs2831699 | rs4819128 | rs7336089 | rs9554641 | rs9561935 |
| rs11910832 | rs1927014 | rs2831702 | rs4819130 | rs733610 | rs9555119 | |
| rs11919425 | rs1927807 | rs2831706 | rs4819201 | rs7336348 | rs9555266 | |
| rs11939712 | rs1927830 | rs2831755 | rs4835587 | rs7336658 | rs9555581 | |
| rs11948061 | rs1930586 | rs2832155 | rs483712 | rs7337326 | rs9555714 | |
| rs11959584 | rs1932917 | rs2832236 | rs4841972 | rs7337382 | rs9556425 | |
| rs11960564 | rs1933187 | rs2832916 | rs4845953 | rs7337528 | rs9560166 | |
| rs12018498 | rs1937443 | rs2833117 | rs486285 | rs7337915 | rs9560339 | |
| rs12020398 | rs1942399 | rs2833123 | rs487812 | rs7338544 | rs9560797 | |
| rs12037545 | rs1942531 | rs2833153 | rs4884402 | rs7339162 | rs9560800 | |
| rs12136961 | rs1942803 | rs2833523 | rs4884905 | rs7339250 | rs9560807 | |
| rs12149 | rs1949593 | rs2833636 | rs4884906 | rs734747 | rs9561254 | |

TABLE 6
Markers used in the present invention

| Marker | Marker | Marker | Marker | Marker | Marker | Marker |
|---|---|---|---|---|---|---|
| number | number | number | number | number | number | number |
| rs12185828 | rs1972598 | rs2834295 | rs4890333 | rs742276 | rs9562045 | rs12584118 |
| rs12287199 | rs1972917 | rs2834297 | rs4890698 | rs743446 | rs9562457 | rs12584427 |
| rs1231048 | rs1979613 | rs2834337 | rs4890876 | rs7441242 | rs9562501 | rs2027667 |
| rs12326252 | rs1980080 | rs2834339 | rs4891097 | rs747781 | rs9562637 | rs2031446 |
| rs12327010 | rs1980942 | rs2834694 | rs4891098 | rs748607 | rs9563028 | rs2836404 |
| rs1236411 | rs1980950 | rs2834709 | rs4891160 | rs7504436 | rs9563770 | rs2836488 |
| rs12420519 | rs1981084 | rs2834712 | rs4891325 | rs7504842 | rs9564167 | rs4941643 |
| rs12427782 | rs1981390 | rs2834756 | rs4891576 | rs7509953 | rs9564355 | rs4941715 |
| rs12428610 | rs1982837 | rs2834782 | rs4891734 | rs7544781 | rs9564535 | rs7735484 |
| rs12428798 | rs1986899 | rs2834796 | rs4907464 | rs754777 | rs9564577 | rs7799930 |
| rs12454023 | rs1988657 | rs2834884 | rs4907552 | rs756040 | rs9564626 | rs9574824 |
| rs12454180 | rs1991753 | rs2834908 | rs4910359 | rs760345 | rs9564747 | rs9574897 |
| rs12454706 | rs1993355 | rs2835035 | rs4911045 | rs7614 | rs9564791 | rs12583161 |
| rs12455429 | rs199667 | rs2835043 | rs4920104 | rs7616178 | rs9565398 | rs12583202 |
| rs12456484 | rs1997353 | rs2835103 | rs4920520 | rs7618973 | rs9565654 | rs2026744 |
| rs12457067 | rs1998956 | rs2835104 | rs492338 | rs762173 | rs9565661 | rs2027605 |
| rs12457191 | rs2000416 | rs2835121 | rs492346 | rs762227 | rs9565968 | rs2836338 |
| rs12458066 | rs2000490 | rs2835169 | rs492597 | rs7624098 | rs9566836 | rs2836358 |
| rs12458637 | rs2000833 | rs2835293 | rs4927236 | rs7624366 | rs9567448 | rs4941183 |
| rs12458713 | rs2004000 | rs2835349 | rs492781 | rs762438 | rs9567700 | rs4941388 |
| rs12482086 | rs2005187 | rs2835567 | rs4939701 | rs7626725 | rs9568497 | rs7728402 |
| rs12482146 | rs2006089 | rs2835695 | rs4939702 | rs7633784 | rs9568684 | rs7733022 |
| rs12482714 | rs200680 | rs2835704 | rs4939735 | rs7634577 | rs9568713 | rs9573927 |
| rs12482786 | rs2009879 | rs2835722 | rs4940009 | rs7639145 | rs9569550 | rs9574740 |
| rs12483578 | rs2012898 | rs2835723 | rs4940235 | rs7639867 | rs9570226 | |
| rs12490235 | rs2012982 | rs2835735 | rs4940498 | rs7651989 | rs9570290 | |
| rs12513430 | rs2013669 | rs2835790 | rs4940563 | rs765557 | rs9570447 | |
| rs12514412 | rs2014509 | rs2835802 | rs4940615 | rs7661729 | rs9571811 | |
| rs1253809 | rs2014678 | rs2835823 | rs4940791 | rs7702862 | rs9571821 | |
| rs1253811 | rs2018093 | rs2835955 | rs4940955 | rs7711972 | rs9572020 | |
| rs12561781 | rs2019006 | rs2835965 | rs4940957 | rs7716283 | rs9572196 | |
| rs12565445 | rs2025951 | rs2835971 | rs4940960 | rs7717101 | rs9572308 | |
| rs125810 | rs2026263 | rs2835975 | rs4941085 | rs7721965 | rs9573824 | |

TABLE 7
Markers used in the present invention

| Marker | Marker | Marker | Marker | Marker | Marker | Marker |
|---|---|---|---|---|---|---|
| number | number | number | number | number | number | number |
| rs12585235 | rs2031546 | rs2836656 | rs4941939 | rs7807853 | rs9574898 | rs12960453 |
| rs12586094 | rs2032313 | rs2836661 | rs4942060 | rs7822979 | rs9574900 | rs12961253 |
| rs12604515 | rs203332 | rs2836706 | rs4942169 | rs7831906 | rs9575364 | rs2104632 |
| rs12604519 | rs2037920 | rs2836837 | rs4942242 | rs7856187 | rs9575369 | rs2111299 |
| rs12605543 | rs2037921 | rs2836840 | rs4942416 | rs786018 | rs9575372 | rs2837751 |
| rs12605917 | rs2039056 | rs2836842 | rs4942486 | rs7914609 | rs9579214 | rs2837801 |
| rs12605932 | rs2039281 | rs2836943 | rs4942642 | rs794185 | rs9581121 | rs525776 |
| rs12606001 | rs2039622 | rs2836956 | rs4942769 | rs7967526 | rs9583190 | rs526057 |
| rs12626853 | rs2043428 | rs2836958 | rs4942830 | rs797517 | rs9583537 | rs7997078 |
| rs12626876 | rs2044800 | rs2836975 | rs4942931 | rs7981995 | rs9583996 | rs7997881 |
| rs12627315 | rs2046845 | rs2836980 | rs4943119 | rs7982563 | rs958687 | rs977660 |
| rs12627610 | rs2051121 | rs2836985 | rs4943694 | rs7982833 | rs9592665 | rs9783885 |
| rs12627745 | rs2051189 | rs2837129 | rs4943696 | rs7983168 | rs9593922 | rs12959212 |
| rs12630707 | rs2051382 | rs2837302 | rs4949256 | rs7983218 | rs9597134 | rs12960451 |
| rs12637291 | rs2057529 | rs2837381 | rs495737 | rs7984225 | rs9600079 | rs2099255 |
| rs12659620 | rs2058276 | rs2837393 | rs496627 | rs7984261 | rs9601268 | rs2100750 |
| rs12724092 | rs2059757 | rs2837395 | rs4986223 | rs7984523 | rs9601567 | rs2837738 |
| rs1274749 | rs2060816 | rs2837399 | rs4989135 | rs7984835 | rs9604328 | rs2837747 |
| rs12756081 | rs2063222 | rs2837403 | rs499416 | rs7986681 | rs9617452 | rs522505 |
| rs1276034 | rs2065280 | rs2837411 | rs4998815 | rs7988095 | rs962267 | rs524566 |
| rs12763013 | rs2065288 | rs2837490 | rs500910 | rs7988209 | rs9634593 | rs7995700 |
| rs128365 | rs2067741 | rs2837494 | rs501062 | rs7989235 | rs9636883 | rs7996275 |
| rs1284419 | rs2068051 | rs2837512 | rs5023173 | rs798963 | rs9636977 | rs970705 |
| rs12858753 | rs2070535 | rs2837529 | rs508151 | rs7989798 | rs9637300 | rs975336 |
| rs12859190 | rs2071754 | rs2837553 | rs509215 | rs7990298 | rs9646522 | |
| rs12864209 | rs2073425 | rs2837592 | rs509741 | rs7992072 | rs9646629 | |
| rs12876644 | rs2076237 | rs2837637 | rs512699 | rs7992416 | rs9647139 | |
| rs12925084 | rs208932 | rs2837655 | rs513775 | rs7993087 | rs9647235 | |
| rs12936110 | rs2090036 | rs2837701 | rs514556 | rs7993804 | rs9647276 | |
| rs12953319 | rs2094186 | rs2837705 | rs514669 | rs7994585 | rs9652107 | |
| rs12955787 | rs2096507 | rs2837712 | rs515391 | rs7994654 | rs9675925 | |
| rs12957246 | rs2096905 | rs2837717 | rs515551 | rs7995283 | rs9676063 | |
| rs12957256 | rs2097096 | rs2837736 | rs515920 | rs7995306 | rs968906 | |

TABLE 8
Markers used in the present invention

| Marker | Marker | Marker | Marker | Marker | Marker | Marker |
|---|---|---|---|---|---|---|
| number | number | number | number | number | number | number |
| rs12961631 | rs2113462 | rs2837865 | rs532095 | rs7997893 | rs9785659 | rs1326056 |
| rs12961741 | rs2115980 | rs2838004 | rs532625 | rs7997966 | rs9785716 | rs13275667 |
| rs12961750 | rs2116378 | rs2838081 | rs535923 | rs7998641 | rs9785897 | rs2187091 |
| rs12962651 | rs211962 | rs2838104 | rs536419 | rs7999126 | rs9786101 | rs2188584 |
| rs12963212 | rs2120204 | rs2838125 | rs537435 | rs7999812 | rs9786111 | rs2849977 |
| rs12963466 | rs212315 | rs2838304 | rs545723 | rs8000390 | rs9786121 | rs2850125 |
| rs12965753 | rs2136681 | rs2838361 | rs550801 | rs8001960 | rs9786140 | rs608382 |
| rs12966281 | rs2137492 | rs2838438 | rs556046 | rs8002541 | rs9786194 | rs608713 |
| rs12966492 | rs214054 | rs2838441 | rs558700 | rs803815 | rs9786276 | rs8091446 |
| rs12967515 | rs214341 | rs2838568 | rs559372 | rs8083067 | rs9786291 | rs8091825 |
| rs12967616 | rs2146442 | rs2838724 | rs560169 | rs8083437 | rs9786386 | rs9909561 |
| rs12968141 | rs2148443 | rs2838799 | rs561418 | rs8083682 | rs9786773 | rs991045 |
| rs12968648 | rs2149436 | rs2838806 | rs569216 | rs8084206 | rs9786824 | rs1325798 |
| rs12969413 | rs2150419 | rs2838813 | rs575936 | rs8084711 | rs9786876 | rs1325968 |
| rs12969725 | rs2151277 | rs2838815 | rs5761308 | rs8084792 | rs9786885 | rs2183557 |
| rs12971228 | rs2154487 | rs2838820 | rs5764891 | rs8085054 | rs9788296 | rs2186557 |
| rs13046156 | rs2154549 | rs2838887 | rs576808 | rs8085056 | rs9789153 | rs2849697 |
| rs13046342 | rs2154550 | rs2838890 | rs578835 | rs8085222 | rs9805596 | rs2849865 |
| rs1304747 | rs2154723 | rs2838893 | rs581394 | rs8086286 | rs9805694 | rs607127 |
| rs13049234 | rs2155797 | rs2839287 | rs582547 | rs8086449 | rs9805804 | rs6072085 |
| rs13049853 | rs2156187 | rs2839377 | rs582853 | rs8086752 | rs9813365 | rs8091123 |
| rs13050660 | rs2156384 | rs2839386 | rs585632 | rs8086807 | rs9818400 | rs8091380 |
| rs13052088 | rs2156650 | rs2839392 | rs591173 | rs8087052 | rs982328 | rs9891988 |
| rs13087163 | rs2160043 | rs2839468 | rs591498 | rs8087127 | rs9828270 | rs990557 |
| rs13152923 | rs2161775 | rs2839470 | rs593340 | rs8087403 | rs9835007 | |
| rs13159598 | rs2166029 | rs2839508 | rs595106 | rs8087551 | rs9837159 | |
| rs13162651 | rs2174524 | rs2839520 | rs596778 | rs8087849 | rs9845467 | |
| rs13163878 | rs2174571 | rs2842906 | rs599551 | rs8088596 | rs984659 | |
| rs13168731 | rs2174896 | rs28468602 | rs599881 | rs8088779 | rs985035 | |
| rs13178296 | rs2178841 | rs2848957 | rs6014601 | rs8088832 | rs985198 | |
| rs13200025 | rs2178848 | rs2848958 | rs602212 | rs8089359 | rs9853755 | |
| rs1323556 | rs2181753 | rs2848961 | rs6047745 | rs8089613 | rs9861671 | |
| rs1325453 | rs2182957 | rs2849253 | rs607020 | rs8090831 | rs9869577 | |

TABLE 9
Markers used in the present invention

| Marker | Marker | Marker | Marker | Marker | Marker | Marker |
|---|---|---|---|---|---|---|
| number | number | number | number | number | number | number |
| rs1328368 | rs2198683 | rs2850542 | rs6108022 | rs8092218 | rs993930 | rs1378492 |
| rs1328926 | rs220128 | rs2852146 | rs612573 | rs8092926 | rs9944568 | rs1378800 |
| rs13304168 | rs220149 | rs2861624 | rs6137476 | rs8094161 | rs9945284 | rs2246422 |
| rs13304202 | rs220171 | rs28649411 | rs614290 | rs8094280 | rs9945648 | rs2247021 |
| rs1331948 | rs220268 | rs287355 | rs616669 | rs8095071 | rs9945969 | rs3121808 |
| rs1331951 | rs2203754 | rs2873580 | rs619542 | rs8095250 | rs9947210 | rs3127637 |
| rs1333023 | rs2205533 | rs2878293 | rs625028 | rs8095514 | rs9947426 | rs6492589 |
| rs1333027 | rs2206747 | rs2878901 | rs625090 | rs8095747 | rs9947829 | rs6506138 |
| rs1333072 | rs2211681 | rs2885243 | rs626519 | rs8096263 | rs9948368 | rs8131559 |
| rs1334384 | rs2211845 | rs2887596 | rs627527 | rs8096542 | rs9948679 | rs8132424 |
| rs1335282 | rs2211869 | rs2892463 | rs628221 | rs8096605 | rs9948733 | rs9959180 |
| rs1335787 | rs2211938 | rs2897977 | rs630706 | rs8096830 | rs9948841 | rs9959555 |
| rs1335788 | rs2211973 | rs2901821 | rs6311 | rs8097023 | rs9948974 | rs1370079 |
| rs13380936 | rs2212624 | rs2921452 | rs632324 | rs8097306 | rs9949020 | rs1377341 |
| rs13381153 | rs2212626 | rs293105 | rs632678 | rs8097433 | rs9949323 | rs2245411 |
| rs13381188 | rs2212809 | rs2933307 | rs632683 | rs8097467 | rs9949565 | rs2246122 |
| rs1340312 | rs2212828 | rs2941782 | rs632986 | rs8097792 | rs9949574 | rs3106603 |
| rs1340333 | rs2217442 | rs2946523 | rs634293 | rs8097822 | rs9949868 | rs3118045 |
| rs1340562 | rs2222370 | rs2953258 | rs634760 | rs8098182 | rs9949882 | rs6492379 |
| rs13433508 | rs2222999 | rs2953261 | rs639862 | rs8098925 | rs9950906 | rs6492586 |
| rs1345492 | rs2223079 | rs2969931 | rs640254 | rs8099616 | rs9951809 | rs8130781 |
| rs1348466 | rs2226356 | rs2993502 | rs641366 | rs8099832 | rs9951893 | rs8131481 |
| rs1349094 | rs2226358 | rs2997116 | rs6426721 | rs8127266 | rs9952107 | rs9958812 |
| rs1349936 | rs2226798 | rs3011522 | rs6439686 | rs8127332 | rs9952148 | rs9958938 |
| rs13503 | rs2226859 | rs3014944 | rs6442180 | rs8127569 | rs9952357 | |
| rs1351407 | rs2236483 | rs3015419 | rs645539 | rs8127634 | rs9952908 | |
| rs13554 | rs2236944 | rs3019879 | rs645699 | rs8128478 | rs9953136 | |
| rs1358368 | rs2241585 | rs304838 | rs6469456 | rs8128523 | rs9954012 | |
| rs1361029 | rs2242661 | rs306395 | rs6483561 | rs8128650 | rs9954208 | |
| rs1361768 | rs2242752 | rs3091601 | rs6490683 | rs8129332 | rs9954439 | |
| rs1364416 | rs2242753 | rs3096835 | rs6490713 | rs8129919 | rs9955860 | |
| rs1365251 | rs2243936 | rs3101866 | rs6490946 | rs8130292 | rs9957425 | |
| rs1369348 | rs2244188 | rs3106556 | rs6491350 | rs8130587 | rs9957591 | |

TABLE 10
Markers used in the present invention

| Marker | Marker | Marker | Marker | Marker | Marker | Marker |
|---|---|---|---|---|---|---|
| number | number | number | number | number | number | number |
| rs1379823 | rs2247221 | rs3171532 | rs6506332 | rs8132865 | rs9959597 | rs1472406 |
| rs1380332 | rs2248218 | rs326046 | rs6506837 | rs8132870 | rs9959723 | rs1473279 |
| rs1382394 | rs2249360 | rs328125 | rs6507196 | rs8133195 | rs9960454 | rs1474092 |
| rs1385338 | rs2250226 | rs329027 | rs6507440 | rs8134612 | rs9961499 | rs1478526 |
| rs1389561 | rs2250494 | rs331018 | rs6507719 | rs8134986 | rs9963406 | rs2284636 |
| rs1390431 | rs2250926 | rs331020 | rs6507783 | rs8181861 | rs9963983 | rs2287434 |
| rs1412819 | rs2251085 | rs334458 | rs6507967 | rs8184900 | rs9964749 | rs2289152 |
| rs1412822 | rs2251210 | rs336214 | rs6508168 | rs825977 | rs9964911 | rs229441 |
| rs1413021 | rs2252046 | rs336279 | rs6508266 | rs844974 | rs9964940 | rs375484 |
| rs1413158 | rs2252776 | rs340966 | rs6508351 | rs844975 | rs9965174 | rs375886 |
| rs1413435 | rs2252828 | rs341237 | rs6508502 | rs844978 | rs9965410 | rs3760582 |
| rs1415019 | rs225383 | rs341499 | rs651029 | rs844986 | rs9965900 | rs377191 |
| rs1417313 | rs225396 | rs341506 | rs651407 | rs844990 | rs9966050 | rs6562599 |
| rs1417907 | rs2254368 | rs347128 | rs6516794 | rs844999 | rs9966798 | rs6562888 |
| rs1421182 | rs2255059 | rs349714 | rs6516819 | rs845015 | rs9967142 | rs6563329 |
| rs1424406 | rs2255332 | rs352247 | rs6517605 | rs845017 | rs9967277 | rs6565830 |
| rs1430378 | rs2256000 | rs35379414 | rs6518100 | rs845018 | rs9967440 | rs892430 |
| rs1430381 | rs2256417 | rs355708 | rs6518252 | rs845022 | rs9967534 | rs894050 |
| rs1437650 | rs2269145 | rs367841 | rs652539 | rs845024 | rs996906 | rs896036 |
| rs1442134 | rs2269161 | rs369906 | rs6550169 | rs845969 | rs9974136 | rs898484 |
| rs1445728 | rs2269173 | rs372883 | rs6550215 | rs857569 | rs997416 | rs9976426 |
| rs1446770 | rs2274328 | rs373037 | rs655209 | rs858044 | rs9974225 | rs9977055 |
| rs1447740 | rs2274403 | rs3736867 | rs6555064 | rs863075 | rs9974317 | rs9977610 |
| rs1451940 | rs2274463 | rs3736972 | rs6561105 | rs864674 | rs9974879 | rs9977815 |
| rs1452670 | rs2274774 | rs3737893 | rs6561326 | rs875625 | rs9974970 | |
| rs1455514 | rs2276218 | rs3742188 | rs6561605 | rs876165 | rs9975304 | |
| rs1455872 | rs2277798 | rs3744877 | rs6561644 | rs877786 | rs9975452 | |
| rs1464236 | rs2279962 | rs3744998 | rs6561709 | rs877856 | rs9975831 | |
| rs1465509 | rs2281767 | rs3746897 | rs6561727 | rs878971 | rs997587 | |
| rs1467756 | rs2284214 | rs3746924 | rs6561900 | rs883868 | rs9976123 | |
| rs1471171 | rs2284514 | rs3751405 | rs6561924 | rs888789 | rs9976168 | |

In the present invention, the biological sample may be blood, plasma, serum, urine, or saliva.

In the present invention, the marker sequences are amplified using a primer represented by a nucleotide sequence of SEQ ID NOs: 1 to 652.

In the present invention, the marker sequences may have genotypes as shown in Table 11 below for each SNP site, and thus any SNP combination (red) cannot provide information useful for prediction of organ transplant rejection, and any SNP combinations (yellow and green) can provide useful information which is entirely determined according to a random distribution of donor-specific and recipient-specific SNP genes.

TABLE 11
Donor/recipient SNP combinations predicted
by analysis using selected marker set

| Donor/recipient | X:X | X:Y | Y:Y |
|---|---|---|---|
| X:X | N | MI | HI |
| X:Y | HI | NI | HI |
| Y:Y | HI | MI | NI |

HI Highly Informative
MI Moderately Informative
NI No Informative

For example, when an SNP marker combination having a minor allele frequency of 45% is used in the analysis, the ratio between each of donor-specific alleles and each of recipient-specific alleles, calculated by the Hardy-Weinberg equilibrium, is represented in Table 12 below.

TABLE 12
Allele ratios predicted by analysis using an SNP marker
combination having a minor allele frequency of 45%

| X = 55% Y = 45% | X:X (30%) | X:Y (49%) | Y:Y (21%) |
|---|---|---|---|
| X:X (30%) | 9.0% | 14.7% | 6.3% |
| X:Y (49%) | 14.7% | 24.0% | 10.3% |
| Y:Y (21%) | 6.3% | 10.3% | 4.4% |

HIGH 37.6%
MEDIUM 25.0%
LOW 37.4%

In the present invention, the step of amplifying the marker sequences may further comprise amplifying all of the markers shown in Tables 1 to 10.

In the present invention, the ratio between the marker sequences might imply the ratio between the amount of each donor-derived marker sequence and the amount of each recipient-derived marker sequence, selected from the list of markers shown in Tables 1 to 10.

The NGS platform that is used in the present invention is optimized for analysis of sequence fragments having a size of 100 bp. Essential factors to be taken into consideration while making a choice of the NGS platform includes the read-lenth that is readable at the same time, basic error rate, analysis speed, and reaction efficiency.

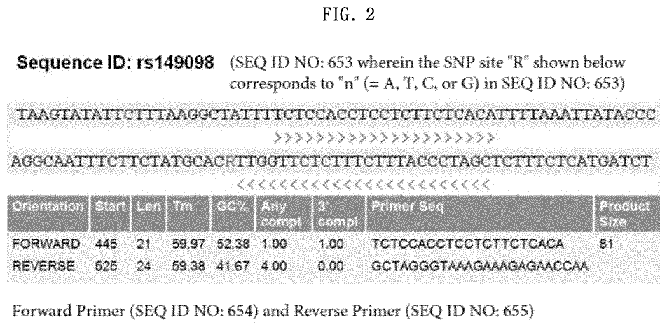

In the present invention, it was shown that when the markers listed in Tables 1 to 10 were amplified in order to optimize the above-described factors, a desired SNP site was within 35 bp from the starting point of sequencing, and the average length of amplified marker sequences were 70 bp (FIG. 2).

Therefore, in the present invention, the amplified marker sequences in the biological sample may be less than 200 bp in length.

The markers that are used in the present invention are bi-allelic SNP sites which are the markers whose positions and expected nucleotide sequences are all known. Thus, when the nucleotide at any position differs from a known nucleotide (for example, A is read in place of the correct nucleotide G/T), it can be counted as an error.

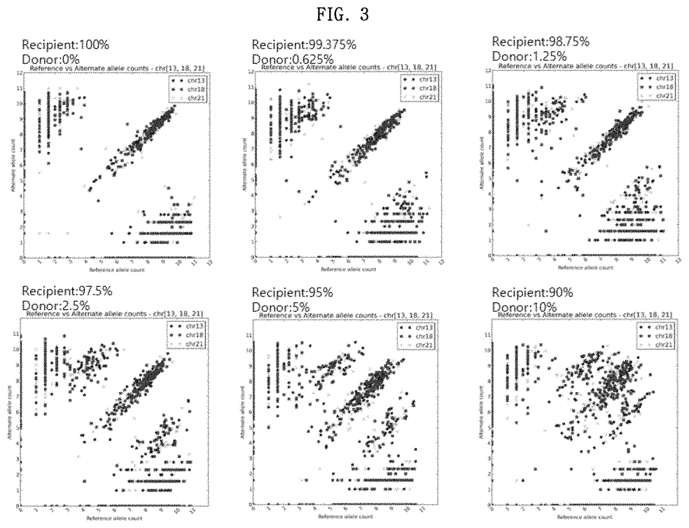

By analyzing 2023 markers as represented in FIG. 3, it was inferred that an error rate could be easily calculated.

In the present invention, the ratio between the marker sequences may be calculated along with a sequencing error rate.

In the present invention, the cutoff values may be reference values from biological sample by a recipient who does not have organ transplant rejection.

Meanwhile, it was found that organ transplant rejection can be predicted by observing a time-dependent change in the amount of donor-derived DNA in a recipient who received an organ.

In another example of the present invention, biological samples were obtained from a recipient, who received an organ, before and immediately after organ transplantation, and then were obtained at certain time intervals after organ transplantation.

The obtained biological samples were analyzed, and as a result, it was observed that the ratio of donor-derived SNP markers were increased when organ transplant rejection occurred.

Therefore, in another aspect, the present invention is directed to a method of predicting organ transplant rejection in a biological sample, obtained from a recipient who received an organ from a donor, by next-generation sequencing (NGS) or digital base amplification, the method comprising the steps of:

non-invasively obtaining a biological sample, which contains donor-derived and recipient-derived cell-free nucleic acid molecules, from a recipient who received an organ from a donor;

amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences in cell-free nucleic acid molecules isolated from the biological sample;

analyzing the amplified sequences by next-generation sequencing (NGS) or digital base amplification;

based on the analysis of the sequences, determining the ratio between each of the donor-derived marker sequences and each of the recipient-derived marker sequences; and

measuring the ratio over time, and predicting whether the recipient will have transplant rejection, graft dysfunction or organ failure when the ratio each of the donor-derived marker sequences increases.

In the present invention, the biological sample may be blood, plasma, serum, urine, or saliva.

In the present invention, the step of amplifying the marker sequences further comprises amplifying all of the markers listed in Tables 1 to 10.

In the present invention, the marker sequences are amplified using a primer represented by a nucleotide sequence of SEQ ID NOs: 1 to 652.

In the present invention, the ratio between the marker sequences might imply the ratio between the amount of each donor-derived marker sequence and the amount of each recipient-derived marker sequence, selected from the markers shown in Tables 1 to 10.

In the present invention, the ratio between the marker sequences may be calculated along with a sequencing error rate.

In the present invention, the amplified marker sequences in the biological sample might be less than 200 bp in length.

In the present invention, the ratio measurement time may be selected from the group consisting of before organ transplantation, immediately after organ transplantation, and one day, two days, one week, one month, two months, three months, one year, two years, and 10 years after organ transplantation.

The present invention is also directed to a computer system comprising a computer readable medium encoded with a plurality of instructions for controlling a computing system to perform an operation of predicting organ transplant rejection in a biological sample, obtained from a recipient who received an organ from a donor, by use of next-generation sequencing (NGS) or digital base amplification,

wherein the biological sample contains donor-derived and recipient-derived cell-free nucleic acid molecules from a recipient who received an organ from a donor, and

the operation comprises the steps of:

receiving data obtained by analyzing rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in the cell-free nucleic acid molecules isolated from the biological sample, by use of next-generation sequencing (NGS) or digital base amplification;

based on the analysis of the sequences, determining the ratio between each of the donor-derived marker sequences and each of the recipient-derived marker sequences;

comparing the ratio with one or more cutoff values; and

based on the comparison, predicting whether or not organ transplant rejection in the recipient will be present.

### EXAMPLES

Hereinafter, the present invention will be described in further detail with reference to examples. It will be obvious to a person having ordinary skill in the art that these examples are for illustrative purposes only and are not to be construed to limit the scope of the present invention. Thus, the substantial scope of the present invention will be defined by the appended claims and equivalents thereof.

### Example 1: Prediction of Organ Transplant Rejection in Artificially Generated Organ Transplant Recipients

1.1: Pretreatment for Preparation and Analysis of Artificial DNA Samples from Organ Transplant Recipients

Male DNA (donor) was mixed with female DNA (recipient) such that the percentage of the male DNA in the female DNA would be 0%, 0.625%, 1.25%, 2.5%, 5% or 10%, thereby preparing artificial organ transplant patient genomic DNA samples.

To perform a TruSeq Custom Amplicon (TSCA) assay (Illumina, USA) using 100 ng of each gDNA, Custom Amplicon was prepared. A heat block was adjusted to 95° C., and 5 μl of each of DNA and CAT (Custom Amplicon Oligo Tube) was added to a 1.7-ml tube. As control reagents, 5 μl of each of ACD1 and ACP1 was also prepared. 40 μl of OHS1 (Oligo Hybridization for Sequencing Reagent 1) was added to each tube and mixed well using a pipette, and each tube was maintained at 95° C. for 1 min, and subjected to oligo hybridization at 40° C. for 80 min subsequently. following this, the temperature was lowered and 45 μl of SW1 (stringent wash 1) reagent was added to an FPU (Filter Plate Unit) plate membrane, followed by centrifugation at 2,400×g and 20° C. for 10 min. The sample tube was subjected to hybridization, spun-down, and the sample was transferred to the FPU plate using a pipette, and then centrifuged at 2,400×g and 20° C. for 2 min. This was followed by the washing of the sample twice with 45 μl of SW1 reagent, and addition of 45 μl of UB1 (Universal Buffer 1) reagent. Subsequently, centrifugation was performed under the same conditions to remove unreacted unbound oligo. For extension-ligation of hybridized oligo, 45 μl of ELM3 (extension-ligation mix 3) was added to the sample covered with a foil and incubated in an incubator at 37° C. for 45 min. After completion of the incubation, the foil was removed, and the sample was centrifuged at 2,400×g for 2 min. Then, 25 μl of 50 mM NaOH was added to the sample which was then pipetted 5 to 6 times using a pipette and incubated at room temperature for 5 minutes. During the incubation, a PMM2/TDP1 (PCR Master Mix 2/TruSeq DNA Polymerase 1) mixture was added to the PCR tube containing P5 and P7 index. After completion of the incubation, 20 μl of DNA diluted in NaOH was added, thereby preparing a total of 50 μl of a PCR amplification sample. The prepared sample was subjected to PCR reaction under the following conditions:

<PCR Conditions>

−95° C., 3 min

−28 cycles

95° C., 30 sec

66° C., 30 sec

72° C., 60 sec

−72° C., 5 min

−Hold at 10° C.

After completion of the PCR cycles, the sample was analyzed using a QIAxcel system for confirmation. The sample was then purified using 60 μl of beads and suspended in 30 μl of resuspension buffer (RS).

1.2: Analysis of Artificial DNA Sample from Organ Transplant Recipient

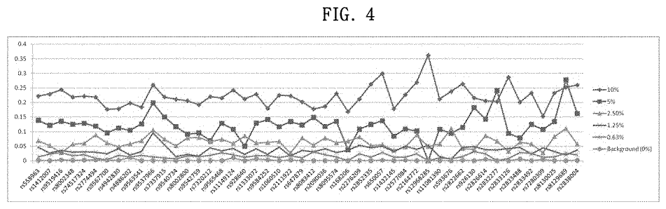

It was observed that the sample could be sequenced using a sequencing system and the corresponding markers could be counted, making it possible to monitor organ transplant rejection through an algorithm and a pipeline (FIGS. 3 and 4).

Allele counts corresponding to the SNP markers identified using next-generation sequencing were graphically shown. On the X-axis, reference allele or major allele counts were expressed, and on the Y-axis, alternate allele or minor allele counts were expressed as log 2 values (FIG. 3). Particularly, because the selected SNP markers were SNPs located at chromosome 13, 18 and 21, these markers were indicated by blue (●), green (▪) and red (x), respectively (FIG. 3).

As represented in Table 13 below, the mixed DNAs may show a total of 9 genotypes.

TABLE 13
Genotypes of mixed (organ transplant patient) DNAs

| | | Donor genotype | | |
|---|---|---|---|---|
| | | AA | AT | TT |
| Recipient | AA | AAAA | AAAT | AATT |
| genotype | AT | ATAA | ATAT | ATTT |
| | TT | TTAA | TTAT | TTTT |

The genotypes of the artificially prepared DNA may appear as AA, AT, TA and TT, and thus have the possibility of eight distributions (AATT and TTAA are regarded as the same genotype). It could be seen that, as the donor-derived DNA increased, the ATAA and ATTT distributions increased at a constant rate (FIG. 3).

As represented in FIG. 3, the distribution of the donor-derived biomarkers changes depending on the degree of mixing of the biomarkers. When this distribution is quantitatively measured and calculated, small amounts of the donor-derived genes present in the recipient's blood can be detected or measured, and when the amount of the donor-derived gene mutation is measured and observed, organ transplant rejection in the recipient can be predicted or observed.

In addition, as represented in Table 14 below, when two DNAs were mixed in different amounts and were actually measured by next-generation sequencing, the amounts of the mixed DNAs could be accurately measured even when present in minute amounts by quantitatively analyzing biomarkers using next-generation sequencing.

TABLE 14
Experimental Ratio and SNP Counting-Based
Ratio of Mixed DNAs

| | Background (0%) | 10% | 5% | 2.50% | 1.25% | 0.63% |
|---|---|---|---|---|---|---|
| Fraction | 0.001395983 | 0.221443 | 0.123786 | 0.063688 | 0.035009 | 0.015802 |
| Homo | | | | | | |
| Fraction | 0.001495645 | 0.110112 | 0.060944 | 0.032425 | 0.017551 | 0.010921 |
| Hetero | | | | | | |
| Actual | | 22.08% | 12.28% | 6.43% | 3.51% | 1.88% |
| Fraction | | | | | | |

In this context, when bases corresponding to AA and aa of artificially mixed donor-derived DNA are counted, values as listed in Table 14 above can be obtained. Although there is a difference of about 2 folds between the experimental value of mixed DNA and the value measured by analysis, this difference may have appeared because the measured DNA amount is not an absolute amount.

As shown in FIG. 4, although markers differ from each other, the use of several markers makes it possible to accurately measure or observe organ transplant rejection.

When the method of the present invention is actually applied to patients, donor-derived DNA can be expressed as a numerical value at varying time points, and organ transplant rejection can be monitored.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### Example 2: Sensitivity Test of the Analysis Method

Assuming the DNA of the AG09387 cell line as the recipient model DNA and assuming the DNA of the GM05381 cell line as the donor DNA, the DNA of the donor (GM05381 cell line) was added to the DNA of the recipient (AG09387 cell line), 10%, 1%, 0.1%, 0.01%, and 0.001% to make the sample for testing sensitivity of analysis method. The DNA used in the experiment was fragmented with an average size of 170 bp, similar to the size of cfDNA using Covaris M220. The library was constructed using a panel to analyze the 334 SNPs shown in Table 1, and analyzed by NGS (next generation sequencing).

Using the NGS method, genotyping of 334 SNPs in Table 15 was performed using the cfDNA of the unmixed beneficiary (AG09387 cell line) and the donor (GM05381 cell line). Then, in the analysis for rejection monitoring, the read counts of SNPs with different genotypes between recipient and donor is calculated and determined the donor cfDNA ratio.

TABLE 15
Marker list used in the test

| | | | | | | | |
|---|---|---|---|---|---|---|---|
| rs6490946 | rs2774494 | rs9285110 | rs876165 | rs4770032 | rs9509249 | rs9509441 | rs9786291 |
| rs6490683 | rs7334546 | rs6490713 | rs9506919 | rs9510334 | rs9510340 | rs1834547 | rs9786140 |
| rs9552733 | rs9510597 | rs9552874 | rs9805694 | rs2148443 | rs7323182 | rs9510775 | rs11096453 |
| rs4770463 | rs9317882 | rs9553022 | rs7992072 | rs736081 | rs1830926 | rs4770597 | rs9786194 |
| rs4770601 | rs943049 | rs9581121 | rs9553390 | rs9551135 | rs4770771 | rs9507577 | rs2058276 |
| rs1886969 | rs306395 | rs9551233 | rs9512046 | rs9507631 | rs9512063 | rs2497654 | rs1358368 |
| rs4771157 | rs1231048 | rs9551406 | rs1778797 | rs9508080 | rs4573787 | rs9579214 | rs9786773 |
| rs1927830 | rs9508327 | rs2892463 | rs1570614 | rs514669 | rs10478 | rs9506275 | rs9786885 |
| rs169332 | rs9508716 | rs7327256 | rs7986681 | rs914532 | rs7339250 | rs4943119 | rs2571764 |
| rs524566 | rs798963 | rs2188584 | rs9534174 | rs9534262 | rs4942486 | rs7330025 | rs3096835 |
| rs2764618 | rs6561924 | rs9570290 | rs11839815 | rs10507416 | rs12876644 | rs1361029 | rs1864469 |
| rs1832265 | rs9564535 | rs1508494 | rs1876583 | rs9564626 | rs9542105 | rs9592665 | rs9786101 |
| rs6562599 | rs9572308 | rs7990298 | rs9542852 | rs12586094 | rs9543171 | rs2031446 | rs3991109 |
| rs9544845 | rs9574740 | rs7988209 | rs9575364 | rs7323558 | rs7325529 | rs4796869 | rs9785716 |
| rs7236653 | rs8095071 | rs1787013 | rs9952107 | rs7231366 | rs7237577 | rs11080646 | rs7067226 |
| rs1284419 | rs9303869 | rs2487257 | rs786018 | rs1030133 | rs968906 | rs10210 | rs7067297 |
| rs892430 | rs12327010 | rs716510 | rs6508351 | rs7228099 | rs12961741 | rs1834545 | rs9785897 |
| rs17533 | rs273696 | rs273701 | rs9303900 | rs1785745 | rs1785739 | rs8096605 | rs1276034 |
| rs1786648 | rs8096542 | rs4800786 | rs16943649 | rs939139 | rs6508502 | rs11083386 | rs17307670 |
| rs2419027 | rs2419041 | rs2729429 | rs17711702 | rs9957591 | rs1026123 | rs336279 | rs9786876 |
| rs9949868 | rs8097023 | rs11081004 | rs878971 | rs1662304 | rs11081037 | rs1790649 | rs9785659 |
| rs2090036 | rs8097433 | rs1390431 | rs12967616 | rs12966492 | rs6507196 | rs4799910 | rs9786121 |
| rs1893455 | rs9947829 | rs8090831 | rs9967142 | rs1567612 | rs1145560 | rs991045 | rs9786111 |
| rs1593579 | rs4133291 | rs9945284 | rs9967440 | rs6506138 | rs11872146 | rs4407150 | rs9786276 |
| rs2222370 | rs2320747 | rs4798412 | rs6506332 | rs694182 | rs341237 | rs9958938 | rs2161775 |
| rs4798479 | rs10502290 | rs2298583 | rs3786355 | rs12185460 | rs16951141 | rs7234111 | rs13304168 |
| rs16951664 | rs602212 | rs8092926 | rs11660737 | rs4239334 | rs8095514 | rs7235989 | rs16980558 |
| rs12606001 | rs2027667 | rs329027 | rs403093 | rs8087127 | rs1023868 | rs2775537 | rs16980586 |
| rs2821796 | rs8134986 | rs12482146 | rs431864 | rs435081 | rs439146 | rs416083 | rs16980588 |
| rs375484 | rs377191 | rs386838 | rs4588273 | rs2013669 | rs2822618 | rs2822648 | rs9786824 |
| rs2242661 | rs2822661 | rs11088040 | rs760345 | rs467140 | rs461853 | rs2822809 | rs765557 |
| rs4817371 | rs1018676 | rs926130 | rs2822965 | rs2822973 | rs2822975 | rs1002460 | rs9786386 |
| rs2256417 | rs2245411 | rs2736084 | rs975336 | rs2823145 | rs2823152 | rs2823169 | rs9983568 |
| rs413285 | rs2205533 | rs2823795 | rs2823809 | rs2823983 | rs2824133 | rs10470201 | rs8129332 |
| rs1014604 | rs2223079 | rs9978408 | rs2226798 | rs2824238 | rs13046156 | rs9984531 | rs229441 |
| rs1102617 | rs8127266 | rs208932 | rs211962 | rs2824376 | rs243594 | rs243601 | rs2826506 |
| rs2183557 | rs1573442 | rs2824762 | rs8132870 | rs2211938 | rs2825516 | rs1491658 | rs2014509 |
| rs863075 | rs1826318 | rs7278137 | rs2825560 | rs2825576 | rs2825578 | rs2825583 | rs11096435 |
| rs2825608 | rs2825610 | rs128365 | rs2825688 | rs1474092 | rs2825824 | rs377685 | rs13304202 |
| rs12482714 | rs2826117 | rs977660 | rs1786427 | rs2826259 | rs1786388 | rs8128523 | rs2826399 |
| rs2826390 | rs2826392 | rs2826396 | rs2096905 | rs13433508 | rs12483578 | | |

The donor cfDNA ratio was set as shown in Table 16 to make 10%, 1%, 0.1%, 0.01% and 0.001%.

TABLE 16
sample information

| | Donor | Recipient |
|---|---|---|
| | cfDNA(%) | cfDNA(%) |
| | (AG09387, XX) | (GM05381, XY) |
| A | 10 | 90 |
| B | 1 | 99 |
| C | 0.1 | 99.9 |
| D | 0.01 | 99.99 |
| E | 0.001 | 99.999 |

TABLE 17
Percentage of donor derived cfDNA in serially mixed samples

| | | Donor allele | Recipient allele [symbol] | Frequency in serially ddcfDNA mixed samples (%) [symbol] | | | | |
|---|---|---|---|---|---|---|---|---|
| [symbol] | RS Number [symbol] | frequency [symbol] | frequency [symbol] | A (10%) [symbol] | B (1%) [symbol] | C (0.1%) [symbol] | D (0.01%) [symbol] | E (0.001%) [symbol] |
| 1 [symbol] | rs6490683 [symbol] | 0.001427891 [symbol] | 0.99963623 [symbol] | 9.7341 [symbol] | 0.8134 [symbol] | 0.0585 [symbol] | 0.0907 [symbol] | 0.0243 [symbol] |
| 2 [symbol] | rs736081 [symbol] | 0.003570897 [symbol] | 0.99959138 [symbol] | 8.3102 [symbol] | 0.9391 [symbol] | 0.1088 [symbol] | 0.0583 [symbol] | 0.0635 [symbol] |
| 3 [symbol] | rs9552733 [symbol] | 0.000734137 [symbol] | 0.99945675 [symbol] | 10.1251 [symbol] | 0.9319 [symbol] | 0.1356 [symbol] | 0.0888 [symbol] | 0.0917 [symbol] |
| 4 [symbol] | rs9551233 [symbol] | 0.002705794 [symbol] | 0.99909903 [symbol] | 9.9187 [symbol] | 1.1021 [symbol] | 0.1548 [symbol] | 0.0730 [symbol] | 0.0895 [symbol] |
| 5 [symbol] | rs9512046 [symbol] | 0.002890933 [symbol] | 0.99905705 [symbol] | 9.2402 [symbol] | 0.9952 [symbol] | 0.1804 [symbol] | 0.1100 [symbol] | 0.1189 [symbol] |
| 6 [symbol] | rs4770463 [symbol] | 0.999339061 [symbol] | 0.00128425 [symbol] | 9.8493 [symbol] | 0.6494 [symbol] | 0.1610 [symbol] | 0.0372 [symbol] | 0.1214 [symbol] |
| 7 [symbol] | rs876165 [symbol] | 0.99905129 [symbol] | 0.00032103 [symbol] | 8.5412 [symbol] | 0.7648 [symbol] | 0.0820 [symbol] | 0.0354 [symbol] | 0.0469 [symbol] |
| 8 [symbol] | rs329027 [symbol] | 0.001454394 [symbol] | 0.99974600 [symbol] | 8.1686 [symbol] | 1.0712 [symbol] | 0.0378 [symbol] | 0.0655 [symbol] | 0.0216 [symbol] |
| 9 [symbol] | rs9958938 [symbol] | 0.001945134 [symbol] | 0.99913218 [symbol] | 9.7477 [symbol] | 0.9026 [symbol] | 0.0085 [symbol] | 0.0953 [symbol] | 0.0927 [symbol] |
| 10 [symbol] | rs602212 [symbol] | 0.996949991 [symbol] | 0.00142005 [symbol] | 9.7177 [symbol] | 1.0685 [symbol] | 0.1582 [symbol] | 0.1292 [symbol] | 0.1315 [symbol] |
| 11 [symbol] | rs1284419 [symbol] | 0.998726309 [symbol] | 0.00123960 [symbol] | 8.0042 [symbol] | 0.9268 [symbol] | 0.1839 [symbol] | 0.1588 [symbol] | 0.1108 [symbol] |
| 12 [symbol] | rs991045 [symbol] | 0.999021814 [symbol] | 0.00015048 [symbol] | 9.3389 [symbol] | 1.0504 [symbol] | 0.0806 [symbol] | 0.0145 [symbol] | 0.0369 [symbol] |
| 13 [symbol] | rs2824133 [symbol] | 0.001182233 [symbol] | 0.99914384 [symbol] | 9.4880 [symbol] | 0.8712 [symbol] | 0.1621 [symbol] | 0.1174 [symbol] | 0.0651 [symbol] |
| 14 [symbol] | rs12482714 [symbol] | 0.002015667 [symbol] | 0.99909941 [symbol] | 11.2350 [symbol] | 1.0765 [symbol] | 0.1420 [symbol] | 0.0792 [symbol] | 0.1237 [symbol] |
| 15 [symbol] | rs9984531 [symbol] | 0.001514975 [symbol] | 0.99842663 [symbol] | 10.1125 [symbol] | 0.8872 [symbol] | 0.2129 [symbol] | 0.2064 [symbol] | 0.1085 [symbol] |
| 16 [symbol] | rs1573442 [symbol] | 0.002131483 [symbol] | 0.99825214 [symbol] | 9.4114 [symbol] | 1.0910 [symbol] | 0.1716 [symbol] | 0.2274 [symbol] | 0.1664 [symbol] |
| 17 [symbol] | rs2211938 [symbol] | 0.999050783 [symbol] | 0.00057557 [symbol] | 10.2037 [symbol] | 1.2259 [symbol] | 0.1428 [symbol] | 0.0138 [symbol] | 0.0542 [symbol] |

| | | | | | |
|---|---|---|---|---|---|
| Average [symbol] | 9.4792 [symbol] | 0.9628 [symbol] | 0.1336 [symbol] | 0.0942 [symbol] | 0.0863 [symbol] |

ddcfDNA, Donor-derived cell-free DNA

The 17 SNPs with different genotypes of beneficiaries and donors were selected and analyzed. The results were similar to the actual mix ratio of 0.1%. The result of 0.01% and 0.001% seems to be higher than the actual mixing ratio because of sequencing error. As a result of t-test analysis of the results of C and D of Table 16, there was a significant difference in p value of 0.044, indicating that the analytical sensitivity of this method was up to 0.1% (D and E were p value of 0.663 which is not meaningful).

### Example 3: Analysis of Kidney Transplant Rejection Patients

Genomic DNA was extracted from donor and recipient blood samples and 334 SNP genotypes shown in Table 15 were analyzed.

CfDNA was extracted from the urine of the recipient collected at 3 days, 7 days, 2 weeks, 1 month, 2 months, 3 months, 6 months after kidney transplantation and analyzed using the panel to analyze 334 SNPs shown in Table 15 and the NGS analysis was performed.

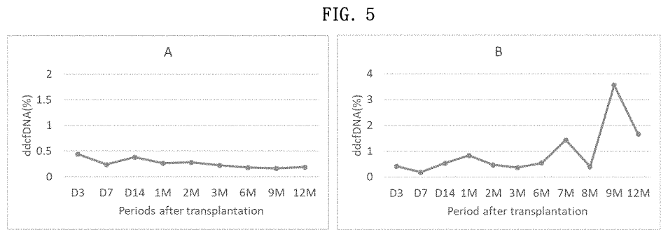

In the case of A patient, in which no specific rejection was observed, the cfDNA ratio of the donor remained constant without significant change as less then 1%, whereas in the case of B patient who had rejection after 2 weeks of kidney transplantation, donor-derived cfDNA was 0.833, 1.43, 3.56, 1.66% at 1M, 7M, 9M, and 12M after organ transplantation. Intermittent responses were also found in the cfDNA analysis (FIG. 5).

### INDUSTRIAL APPLICABILITY

The method of the present invention is useful for non-invasive prediction and monitoring of organ transplant rejection, and thus it has a high industrial applicability.

What is claimed is:

1. A method of predicting organ transplant rejection in a biological sample, obtained from a recipient who received an organ from a donor, by next-generation sequencing (NGS) or digital base amplification, the method comprising the steps of:
obtaining a biological sample non-invasively, which contains donor-derived and recipient-derived cell-free nucleic acid molecules, from a recipient who received an organ from a donor;
amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in cell-free nucleic acid molecules isolated from the biological sample;
analyzing the amplified sequences by next-generation sequencing (NGS) or digital base amplification;
based on the analysis of the sequences, determining the ratio between each of the donor-derived marker sequences and each of the recipient-derived marker sequences; and
comparing the ratio with one or more cutoff values.

2. The method of claim 1, wherein the biological sample is blood, plasma, serum, urine, or saliva.

3. The method of claim 1, wherein the marker sequences are amplified using a primer represented by a nucleotide sequence of SEQ ID NOs: 1 to 652.

4. The method of claim 1, wherein the ratio between the marker sequences is calculated along with a sequencing error rate.

5. The method of claim 1, wherein the amplified marker sequences in the biological sample are less than 200 bp in length.

6. The method of claim 1, wherein the cutoff values are reference values established from biological sample by a recipient who does not have organ transplant rejection.

7. A method of predicting organ transplant rejection in a biological sample, obtained from a recipient who received an organ from a donor, by next-generation sequencing (NGS) or digital base amplification, the method comprising the steps of:
obtaining a biological sample non-invasively, which contains donor-derived and recipient-derived cell-free nucleic acid molecules, from a recipient who received an organ from a donor;
amplifying rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences in cell-free nucleic acid molecules isolated from the biological sample;
analyzing the amplified sequences by next-generation sequencing (NGS) or digital base amplification;
based on the analysis of the sequences, determining the ratio between each of the donor-derived marker sequences and each of the recipient-derived marker sequences; and
measuring the ratio over time, and predicting whether the recipient will have transplant rejection, graft dysfunction or organ failure when the ratio each of the donor-derived marker sequences increases.

8. The method of claim 7, wherein the biological sample is blood, plasma, serum, urine, or saliva.

9. The method of claim 7, wherein the marker sequences are amplified using a primer represented by a nucleotide sequence of SEQ ID NOs: 1 to 652.

10. The method of claim 7, wherein the ratio between the marker sequences is calculated along with a sequencing error rate.

11. The method of claim 7, wherein the amplified marker sequences in the biological sample are less than 200 bp in length.

12. The method of claim 7, wherein the ratio measurement time is selected from the group consisting of before organ transplantation, immediately after organ transplantation, and one day, two days, one week, one month, two months, three months, one year, two years, and 10 years after organ transplantation.

13. A computer system comprising a computer readable medium encoded with a plurality of instructions for controlling a computing system to perform an operation of predicting organ transplant rejection in a biological sample, obtained from a recipient who received an organ from a donor, by use of next-generation sequencing (NGS) or digital base amplification,
wherein the biological sample contains donor-derived and recipient-derived cell-free nucleic acid molecules from a recipient who received an organ from a donor, and
wherein the operation comprises the steps of:
receiving data obtained by analyzing rs6490946, rs6490683, rs9552733, rs4770463, rs4770601, rs1886969, rs4771157, rs1927830, rs169332, rs524566, rs2764618, rs1832265, rs6562599, rs9544845, rs7236653, rs1284419, rs892430, rs17533, rs1786648, rs2419027, rs9949868, rs2090036, rs1893455, rs1593579, rs2222370, rs4798479, rs16951664, rs12606001, rs2821796, rs375484, rs2242661, rs4817371, rs2256417, rs413285, rs1014604, rs1102617, rs2183557, rs863075, rs2825608, rs12482714, rs2826390, rs2774494, rs7334546, rs9510597, rs9317882, rs943049, rs306395, rs1231048, rs9508327, rs9508716, rs798963, rs6561924, rs9564535, rs9572308, rs9574740, rs8095071, rs9303869, rs12327010, rs273696, rs8096542, rs2419041, rs8097023, rs8097433, rs9947829, rs4133291, rs2320747, rs10502290, rs602212, rs2027667, rs8134986, rs377191, rs2822661, rs1018676, rs2245411, rs2205533, rs2223079, rs8127266, rs1573442, rs1826318, rs2825610, rs2826117, rs2826392, rs9285110, rs6490713, rs9552874, rs9553022, rs9581121, rs9551233, rs9551406, rs2892463, rs7327256, rs2188584, rs9570290, rs1508494, rs7990298, rs7988209, rs1787013, rs2487257, rs716510, rs273701, rs4800786, rs2729429, rs11081004, rs1390431, rs8090831, rs9945284, rs4798412, rs2298583, rs8092926, rs329027, rs12482146, rs386838, rs11088040, rs926130, rs2736084, rs2823795, rs9978408, rs208932, rs2824762, rs7278137, rs128365, rs977660, rs2826396, rs876165, rs9506919, rs9805694, rs7992072, rs9553390, rs9512046, rs1778797, rs1570614, rs7986681, rs9534174, rs11839815, rs1876583, rs9542852, rs9575364, rs9952107, rs786018, rs6508351, rs9303900, rs16943649, rs17711702, rs878971, rs12967616, rs9967142, rs9967440, rs6506332, rs3786355, rs11660737, rs403093, rs431864, rs4588273, rs760345, rs2822965, rs975336, rs2823809, rs2226798, rs211962, rs8132870, rs2825560, rs2825688, rs1786427, rs2096905, rs4770032, rs9510334, rs2148443, rs736081, rs9551135, rs9507631, rs9508080, rs514669, rs914532, rs9534262, rs10507416, rs9564626, rs12586094, rs7323558, rs7231366, rs1030133, rs7228099, rs1785745, rs939139, rs9957591, rs1662304, rs12966492, rs1567612, rs6506138, rs694182, rs12185460, rs4239334, rs8087127, rs435081, rs2013669, rs467140, rs2822973, rs2823145, rs2823983, rs2824238, rs2824376, rs2211938, rs2825576, rs1474092, rs2826259, rs13433508, rs9509249, rs9510340, rs7323182, rs1830926, rs4770771, rs9512063, rs4573787, rs10478, rs7339250, rs4942486, rs12876644, rs9542105, rs9543171, rs7325529, rs7237577, rs968906, rs12961741, rs1785739, rs6508502, rs1026123, rs11081037, rs6507196, rs1145560, rs11872146, rs341237, rs16951141, rs8095514, rs1023868, rs439146, rs2822618, rs461853, rs2822975, rs2823152, rs2824133, rs13046156, rs243594, rs2825516, rs2825578, rs2825824, rs1786388, rs12483578, rs9509441, rs1834547, rs9510775, rs4770597, rs9507577, rs2497654, rs9579214, rs9506275, rs4943119, rs7330025, rs1361029, rs9592665, rs2031446, rs4796869, rs11080646, rs10210, rs1834545, rs8096605, rs11083386, rs336279, rs1790649, rs4799910, rs991045, rs4407150, rs9958938, rs7234111, rs7235989, rs2775537, rs416083, rs2822648, rs2822809, rs1002460, rs2823169, rs10470201, rs9984531, rs243601, rs1491658, rs2825583, rs377685, rs8128523, rs9786291, rs9786140, rs11096453, rs9786194, rs2058276, rs1358368, rs9786773, rs9786885, rs2571764, rs3096835, rs1864469, rs9786101, rs3991109, rs9785716, rs7067226, rs7067297, rs9785897, rs1276034, rs17307670, rs9786876, rs9785659, rs9786121, rs9786111, rs9786276, rs2161775, rs13304168, rs16980558, rs16980586, rs16980588, rs9786824, rs765557, rs9786386, rs9983568, rs8129332, rs229441, rs2826506, rs2014509, rs11096435, rs13304202 and rs2826399 marker sequences, in the cell-free nucleic acid molecules isolated from the biological sample, by use of next-generation sequencing (NGS) or digital base amplification;
based on the analysis of the sequences, determining the ratio between each of the donor-derived marker sequences and each of the recipient-derived marker sequences;
comparing the ratio with one or more cutoff values; and
based on the comparison, predicting whether or not organ transplant rejection in the recipient will be present.


SEQUENCE LISTING (655 sequences)

SEQ ID NO: 1 - DNA, 24 bp, Artificial Sequence
  rs6490946_F
cacacaccattaccaatggttctc

SEQ ID NO: 2 - DNA, 22 bp, Artificial Sequence
  rs6490946_R
cctcaccatcaccaaccttgag

SEQ ID NO: 3 - DNA, 24 bp, Artificial Sequence
  rs2774494_F
ccctttcttgggtggtagaattca

SEQ ID NO: 4 - DNA, 25 bp, Artificial Sequence
  rs2774494_R
cacctgttgctgttttcatcacaaa

SEQ ID NO: 5 - DNA, 25 bp, Artificial Sequence
  rs9285110_F
gtttgagcgattacacagacaacac

SEQ ID NO: 6 - DNA, 29 bp, Artificial Sequence
  rs9285110_R
atgggatgattgaaaacaacaataacacc

SEQ ID NO: 7 - DNA, 23 bp, Artificial Sequence
  rs876165_F
tgggcctttcgcttttctttcta

SEQ ID NO: 8 - DNA, 24 bp, Artificial Sequence
  rs876165_R
ggccatttctccttccaaagtgaa

SEQ ID NO: 9 - DNA, 23 bp, Artificial Sequence
  rs4770032_F
agttggaaccctcaagggctata

SEQ ID NO: 10 - DNA, 30 bp, Artificial Sequence
  rs4770032_R
ccctaagggtctatttctaaagtctgtttt

SEQ ID NO: 11 - DNA, 25 bp, Artificial Sequence
  rs9509249_F
gaaggagaagaaatgcagacttcca

SEQ ID NO: 12 - DNA, 30 bp, Artificial Sequence
  rs9509249_R
tcaaatgtgttgtaatctccaaacaaagtc

SEQ ID NO: 13 - DNA, 28 bp, Artificial Sequence
  rs9509441_F
attctagattagacataggcaagcacat

SEQ ID NO: 14 - DNA, 28 bp, Artificial Sequence
  rs9509441_R
aaaaaggttttagaactccctgaatgga

SEQ ID NO: 15 - DNA, 30 bp, Artificial Sequence
  rs6490683_F
agacctagaagcagagatgttattgattct

SEQ ID NO: 16 - DNA, 28 bp, Artificial Sequence
  rs6490683_R
ctaaaaccaccacacagactcacacaca

SEQ ID NO: 17 - DNA, 22 bp, Artificial Sequence
  rs7334546_F
agaggagtccaggacttggaag

SEQ ID NO: 18 - DNA, 22 bp, Artificial Sequence
  rs7334546_R
cgtggccacaaacatctcttct

SEQ ID NO: 19 - DNA, 22 bp, Artificial Sequence
  rs6490713_F
agatggctcactcactgagaga

SEQ ID NO: 20 - DNA, 22 bp, Artificial Sequence
  rs6490713_R
cccaagtggtgtgacgttaact

SEQ ID NO: 21 - DNA, 24 bp, Artificial Sequence
  rs9506919_F
tcagcaatgtcgaaactgtactga

SEQ ID NO: 22 - DNA, 22 bp, Artificial Sequence
  rs9506919_R
atccactaccgcaccacacact

SEQ ID NO: 23 - DNA, 31 bp, Artificial Sequence
  rs9510334_F
tgcactcttatttatgtttttccatttgagc

SEQ ID NO: 24 - DNA, 24 bp, Artificial Sequence
  rs9510334_R
ggacaagaataagccacgtgtagg

SEQ ID NO: 25 - DNA, 27 bp, Artificial Sequence
  rs9510340_F
ccaccattatcacaaatacctcttgca

SEQ ID NO: 26 - DNA, 29 bp, Artificial Sequence
  rs9510340_R
cagatcaacattacatatgcatcaccaat

SEQ ID NO: 27 - DNA, 22 bp, Artificial Sequence
  rs1834547_F
gaggaagaggtggcattctaca

SEQ ID NO: 28 - DNA, 26 bp, Artificial Sequence
  rs1834547_R
ctatactcttttcctggaagcaacca

SEQ ID NO: 29 - DNA, 23 bp, Artificial Sequence
  rs9552733_F
ggatctcggagagtttctccctt

SEQ ID NO: 30 - DNA, 22 bp, Artificial Sequence
  rs9552733_R
cccaaattgtcgggtcctctat

SEQ ID NO: 31 - DNA, 27 bp, Artificial Sequence
  rs9510597_F
agctcaaaaataaactggctacaactt

SEQ ID NO: 32 - DNA, 28 bp, Artificial Sequence
  rs9510597_R
caaaaagttcctcaagacaaaacctgat

SEQ ID NO: 33 - DNA, 22 bp, Artificial Sequence
  rs9552874_F
gagcttccaacagttcctccat

SEQ ID NO: 34 - DNA, 30 bp, Artificial Sequence
  rs9552874_R
tcctggtttttattagatgctctcagaaat

SEQ ID NO: 35 - DNA, 24 bp, Artificial Sequence
  rs9805694_F
cttcaatctacagcacacccttgt

SEQ ID NO: 36 - DNA, 23 bp, Artificial Sequence
  rs9805694_R
caaagatgcatgggtagagcaga

SEQ ID NO: 37 - DNA, 22 bp, Artificial Sequence
  rs2148443_F
ggacacatgagactgcactttc

SEQ ID NO: 38 - DNA, 29 bp, Artificial Sequence
  rs2148443_R
agaaaagcatttacagtgaatatggagct

SEQ ID NO: 39 - DNA, 28 bp, Artificial Sequence
  rs7323182_F
agaactagttactggatttgtcgattcc

SEQ ID NO: 40 - DNA, 30 bp, Artificial Sequence
  rs7323182_R
cctataatgtcggaccacatattttcaaca

SEQ ID NO: 41 - DNA, 29 bp, Artificial Sequence
  rs9510775_F
gcaacatagcaagattctgacactaaaat

SEQ ID NO: 42 - DNA, 24 bp, Artificial Sequence
  rs9510775_R
cccatccttcaagtcctccacata

SEQ ID NO: 43 - DNA, 22 bp, Artificial Sequence
  rs4770463_F
gggcagctagtcaagcagaata

SEQ ID NO: 44 - DNA, 26 bp, Artificial Sequence
  rs4770463_R
caggacttcttgcactatgtctaagg

SEQ ID NO: 45 - DNA, 22 bp, Artificial Sequence
  rs9317882_F
aggccctgtagtgtagtggtta

SEQ ID NO: 46 - DNA, 30 bp, Artificial Sequence
  rs9317882_R
gatgtgatctgtgttcacgacaattttaaa

SEQ ID NO: 47 - DNA, 23 bp, Artificial Sequence
  rs9553022_F
tgagtatctgaggcagcagagaa

SEQ ID NO: 48 - DNA, 25 bp, Artificial Sequence
  rs9553022_R
tggtttccacataagcaacctttga

SEQ ID NO: 49 - DNA, 26 bp, Artificial Sequence
  rs7992072_F
gaggaagaagaaggacaagatgactc

SEQ ID NO: 50 - DNA, 23 bp, Artificial Sequence
  rs7992072_R
gcctaggcaccctctagctttta

SEQ ID NO: 51 - DNA, 24 bp, Artificial Sequence
  rs736081_F
gcaacattggccaacttagaaacg

SEQ ID NO: 52 - DNA, 28 bp, Artificial Sequence
  rs736081_R
aatacactggaccctaaatgaaactctg

SEQ ID NO: 53 - DNA, 30 bp, Artificial Sequence
  rs1830926_F
tccaattaaatgctctagataccacctttc

SEQ ID NO: 54 - DNA, 29 bp, Artificial Sequence
  rs1830926_R
atcaggaaatgaattggcactttcatatt

SEQ ID NO: 55 - DNA, 22 bp, Artificial Sequence
  rs4770597_F
tgtgcagcctatacagaccaga

SEQ ID NO: 56 - DNA, 22 bp, Artificial Sequence
  rs4770597_R
ccctcatcttcaggcatcccta

SEQ ID NO: 57 - DNA, 23 bp, Artificial Sequence
  rs4770601_F
gggaactgtaaggaaatgccagt

SEQ ID NO: 58 - DNA, 29 bp, Artificial Sequence
  rs4770601_R
tgaaagaccattcagaaactcagatgaat

SEQ ID NO: 59 - DNA, 28 bp, Artificial Sequence
  rs943049_F
ctttctcatctgagagagttggtattcc

SEQ ID NO: 60 - DNA, 22 bp, Artificial Sequence
  rs943049_R
gcacctgccaaaagaaacatgg

SEQ ID NO: 61 - DNA, 22 bp, Artificial Sequence
  rs9581121_F
ctgacccatcctgacaccaatc

SEQ ID NO: 62 - DNA, 28 bp, Artificial Sequence
  rs9581121_R
tatacattctgctgacagaagcacatac

SEQ ID NO: 63 - DNA, 22 bp, Artificial Sequence
  rs9553390_F
ccaaattcgggtcttcctcact

SEQ ID NO: 64 - DNA, 22 bp, Artificial Sequence
  rs9553390_R
cgaagccttgggacaagtagag

SEQ ID NO: 65 - DNA, 22 bp, Artificial Sequence
  rs9551135_F
caaacaatccagctgtgcagtt

SEQ ID NO: 66 - DNA, 22 bp, Artificial Sequence
  rs9551135_R
caaggatgagactcagaccaca

SEQ ID NO: 67 - DNA, 30 bp, Artificial Sequence
  rs4770771_F
gtgggaagattacttgaatttaggagttca

SEQ ID NO: 68 - DNA, 24 bp, Artificial Sequence
  rs4770771_R
tgggaaaagagtctgatagcagga

SEQ ID NO: 69 - DNA, 24 bp, Artificial Sequence
  rs9507577_F
attaactcttgagtgggcttgctt

SEQ ID NO: 70 - DNA, 29 bp, Artificial Sequence
  rs9507577_R
tatgattttgaaataccatggaccacaca

SEQ ID NO: 71 - DNA, 28 bp, Artificial Sequence
  rs1886969_F
attgaagaaattgggcctttgttttcat

SEQ ID NO: 72 - DNA, 30 bp, Artificial Sequence
  rs1886969_R
acttgtttgaaatgtcccttatcacatgat

SEQ ID NO: 73 - DNA, 22 bp, Artificial Sequence
  rs306395_F
gctcagttgaggcgtaggattt

SEQ ID NO: 74 - DNA, 29 bp, Artificial Sequence
  rs306395_R
tttttcatggtcagtcttactctgttttg

SEQ ID NO: 75 - DNA, 22 bp, Artificial Sequence
  rs9551233_F
aagaggagctgcctgcaaatta

SEQ ID NO: 76 - DNA, 30 bp, Artificial Sequence
  rs9551233_R
tttttcccatagaactactaggtacgtatg

SEQ ID NO: 77 - DNA, 22 bp, Artificial Sequence
  rs9512046_F
ctgcagcgaggagaatgggaga

SEQ ID NO: 78 - DNA, 29 bp, Artificial Sequence
  rs9512046_R
tctccacagaacgaagtttatgtttcttt

SEQ ID NO: 79 - DNA, 24 bp, Artificial Sequence
  rs9507631_F
gtgaccctgtagctacttaggaga

SEQ ID NO: 80 - DNA, 25 bp, Artificial Sequence
  rs9507631_R
agagttgactttctctgtcccttga

SEQ ID NO: 81 - DNA, 28 bp, Artificial Sequence
  rs9512063_F
tcagtacacttctggcttcatattgttg

SEQ ID NO: 82 - DNA, 27 bp, Artificial Sequence
  rs9512063_R
gataacattgtgcataggctggaaatt

SEQ ID NO: 83 - DNA, 30 bp, Artificial Sequence
  rs2497654_F
caatgtgctacagtacacaaatagatacac

SEQ ID NO: 84 - DNA, 26 bp, Artificial Sequence
  rs2497654_R
aaccataacagttggttagattgcct

SEQ ID NO: 85 - DNA, 30 bp, Artificial Sequence
  rs4771157_F
gtttaaacaaaaggtttctaagatgcctgt

SEQ ID NO: 86 - DNA, 28 bp, Artificial Sequence
  rs4771157_R
tcctcacaataactctgaaaaatcagca

SEQ ID NO: 87 - DNA, 23 bp, Artificial Sequence
  rs1231048_F
catcctgtcacccaggaaagaaa

SEQ ID NO: 88 - DNA, 30 bp, Artificial Sequence
  rs1231048_R
ggtttaggtagaagcagttaataacaacct

SEQ ID NO: 89 - DNA, 23 bp, Artificial Sequence
  rs9551406_F
ccagccaggtgtgtttcttcatt

SEQ ID NO: 90 - DNA, 30 bp, Artificial Sequence
  rs9551406_R
cgttcatgcaatttcatctaaagttgaaga

SEQ ID NO: 91 - DNA, 23 bp, Artificial Sequence
  rs1778797_F
ggtggaaaggatgcaagagagaa

SEQ ID NO: 92 - DNA, 29 bp, Artificial Sequence
  rs1778797_R
tgttacaaatcaagggtagatggtcaaat

SEQ ID NO: 93 - DNA, 23 bp, Artificial Sequence
  rs9508080_F
gcccagccaaggactcttaaaag

SEQ ID NO: 94 - DNA, 26 bp, Artificial Sequence
  rs9508080_R
tggagaaagatatgagagacagccaa

SEQ ID NO: 95 - DNA, 29 bp, Artificial Sequence
  rs4573787_F
taaagggagaaacatagttaagggagagt

SEQ ID NO: 96 - DNA, 30 bp, Artificial Sequence
  rs4573787_R
aaaaagaattccggatccttagagagaatt

SEQ ID NO: 97 - DNA, 28 bp, Artificial Sequence
  rs9579214_F
gggaagaggcttagatattttctgtagt

SEQ ID NO: 98 - DNA, 27 bp, Artificial Sequence
  rs9579214_R
ggcctattctatccataaggacactga

SEQ ID NO: 99 - DNA, 24 bp, Artificial Sequence
  rs1927830_F
gagggagtgtggcttactatttca

SEQ ID NO: 100 - DNA, 26 bp, Artificial Sequence
  rs1927830_R
acgttccatatttatcaggcaatgga

SEQ ID NO: 101 - DNA, 23 bp, Artificial Sequence
  rs9508327_F
cacagggaggaggaggatctaaa

SEQ ID NO: 102 - DNA, 24 bp, Artificial Sequence
  rs9508327_R
ggacagaactagagaaggcactca

SEQ ID NO: 103 - DNA, 29 bp, Artificial Sequence
  rs2892463_F
tgctccatagatacaaaggtaaatgacag

SEQ ID NO: 104 - DNA, 26 bp, Artificial Sequence
  rs2892463_R
gttttccttagcatacccttctgcta

SEQ ID NO: 105 - DNA, 22 bp, Artificial Sequence
  rs1570614_F
agcccacctccataccttacat

SEQ ID NO: 106 - DNA, 28 bp, Artificial Sequence
  rs1570614_R
gctttaccagtagaaggactcttggtat

SEQ ID NO: 107 - DNA, 22 bp, Artificial Sequence
  rs514669_F
ctagcaacagtgggcagcaaca

SEQ ID NO: 108 - DNA, 22 bp, Artificial Sequence
  rs514669_R
ctcccacagaacatacttggca

SEQ ID NO: 109 - DNA, 22 bp, Artificial Sequence
  rs10478_F
gtgtccactcacagccatgtta

SEQ ID NO: 110 - DNA, 29 bp, Artificial Sequence
  rs10478_R
tccatctttctttctctagcaaagtgttt

SEQ ID NO: 111 - DNA, 30 bp, Artificial Sequence
  rs9506275_F
ttctgactggcaatagtttgcaaaataatc

SEQ ID NO: 112 - DNA, 27 bp, Artificial Sequence
  rs9506275_R
tcctgaattctcagtttcgaatagtgc

SEQ ID NO: 113 - DNA, 29 bp, Artificial Sequence
  rs169332_F
accatgaccataaaaagcacataaaaagg

SEQ ID NO: 114 - DNA, 30 bp, Artificial Sequence
  rs169332_R
agctgtaaactttctggtacgtatcttttt

SEQ ID NO: 115 - DNA, 26 bp, Artificial Sequence
  rs9508716_F
gccaggttatacttctgcaattacag

SEQ ID NO: 116 - DNA, 25 bp, Artificial Sequence
  rs9508716_R
agattagacgttgtttgctcactgt

SEQ ID NO: 117 - DNA, 24 bp, Artificial Sequence
  rs7327256_F
gctttttgaagagggtaggatgga

SEQ ID NO: 118 - DNA, 23 bp, Artificial Sequence
  rs7327256_R
gtgtggaagtgacaatagtgggt

SEQ ID NO: 119 - DNA, 28 bp, Artificial Sequence
  rs7986681_F
aacaccgattttctctgtactctattgg

SEQ ID NO: 120 - DNA, 22 bp, Artificial Sequence
  rs7986681_R
gcctaacctggagcctaaggat

SEQ ID NO: 121 - DNA, 27 bp, Artificial Sequence
  rs914532_F
tgtgaatcagatttggagttctaggga

SEQ ID NO: 122 - DNA, 27 bp, Artificial Sequence
  rs914532_R
gaaaatttattctgtggcatccctgtt

SEQ ID NO: 123 - DNA, 30 bp, Artificial Sequence
  rs7339250_F
gatctctctatgtagtgcaagataaagctt

SEQ ID NO: 124 - DNA, 26 bp, Artificial Sequence
  rs7339250_R
tgcctggtctatgacagagactaaaa

SEQ ID NO: 125 - DNA, 23 bp, Artificial Sequence
  rs4943119_F
cgactggaaatcatctccagaga

SEQ ID NO: 126 - DNA, 23 bp, Artificial Sequence
  rs4943119_R
tcctttccatccgtggttgtatc

SEQ ID NO: 127 - DNA, 28 bp, Artificial Sequence
  rs524566_F
tgaagtttgcaagaccaatagaatagca

SEQ ID NO: 128 - DNA, 30 bp, Artificial Sequence
  rs524566_R
gccatgtttttatcatttttgtcactttcc

SEQ ID NO: 129 - DNA, 29 bp, Artificial Sequence
  rs798963_F
tctttttaattgcctggaaattcacagtc

SEQ ID NO: 130 - DNA, 29 bp, Artificial Sequence
  rs798963_R
atgcaacttggagaatctttattaaccct

SEQ ID NO: 131 - DNA, 24 bp, Artificial Sequence
  rs2188584_F
ccaatcacactccctaactgatga

SEQ ID NO: 132 - DNA, 30 bp, Artificial Sequence
  rs2188584_R
gctctgaggtattctgaagagaaagatttt

SEQ ID NO: 133 - DNA, 29 bp, Artificial Sequence
  rs9534174_F
ggagacgatgaaaataatgtctcataggt

SEQ ID NO: 134 - DNA, 27 bp, Artificial Sequence
  rs9534174_R
acactctgaagaaaagggaaagttcat

SEQ ID NO: 135 - DNA, 30 bp, Artificial Sequence
  rs9534262_F
atgctcagcaatgaagtttttatcagtatg

SEQ ID NO: 136 - DNA, 28 bp, Artificial Sequence
  rs9534262_R
cctgtattttagttgaagaagcaccctt

SEQ ID NO: 137 - DNA, 30 bp, Artificial Sequence
  rs4942486_F
catttataattggaggatcatttttgccgt

SEQ ID NO: 138 - DNA, 26 bp, Artificial Sequence
  rs4942486_R
gttccttttgttcagcagattccatg

SEQ ID NO: 139 - DNA, 28 bp, Artificial Sequence
  rs7330025_F
ggtaaatggatatgtgtctttgctaggt

SEQ ID NO: 140 - DNA, 29 bp, Artificial Sequence
  rs7330025_R
tgtaacattcgtctgacaaatagacgaaa

SEQ ID NO: 141 - DNA, 23 bp, Artificial Sequence
  rs2764618_F
gaaaacgtcctggctgctaagta

SEQ ID NO: 142 - DNA, 22 bp, Artificial Sequence
  rs2764618_R
accgtgctcgaccatgtatttc

SEQ ID NO: 143 - DNA, 28 bp, Artificial Sequence
  rs6561924_F
tctaatggaaggcttgtaattggttctg

SEQ ID NO: 144 - DNA, 22 bp, Artificial Sequence
  rs6561924_R
ctggcctaccctcacctatttt

SEQ ID NO: 145 - DNA, 23 bp, Artificial Sequence
  rs9570290_F
ctgtgtacaggttgtgggtaaga

SEQ ID NO: 146 - DNA, 30 bp, Artificial Sequence
  rs9570290_R
gctcacgttattagggaagaaaaattcatc

SEQ ID NO: 147 - DNA, 26 bp, Artificial Sequence
  rs11839815_F
accacacccagcttatgatatgtttt

SEQ ID NO: 148 - DNA, 30 bp, Artificial Sequence
  rs11839815_R
agcctggttcatctaataaaagaaagctaa

SEQ ID NO: 149 - DNA, 24 bp, Artificial Sequence
  rs10507416_F
agatggataaggaaggctgagaca

SEQ ID NO: 150 - DNA, 28 bp, Artificial Sequence
  rs10507416_R
tggtctaaagttttctcttaacaagcca

SEQ ID NO: 151 - DNA, 23 bp, Artificial Sequence
  rs12876644_F
agatatgggcagcattgtttgac

SEQ ID NO: 152 - DNA, 25 bp, Artificial Sequence
  rs12876644_R
tgacttcaaactctcctctgagtca

SEQ ID NO: 153 - DNA, 22 bp, Artificial Sequence
  rs1361029_F
gtaatagccaggctctgacaca

SEQ ID NO: 154 - DNA, 23 bp, Artificial Sequence
  rs1361029_R
tcttgagccatgctacacaaagt

SEQ ID NO: 155 - DNA, 23 bp, Artificial Sequence
  rs1832265_F
aaggtgtgtagagaggacttggt

SEQ ID NO: 156 - DNA, 24 bp, Artificial Sequence
  rs1832265_R
cccatttgtttaggtgtgctctca

SEQ ID NO: 157 - DNA, 22 bp, Artificial Sequence
  rs9564535_F
cccaagccactggtctactttc

SEQ ID NO: 158 - DNA, 22 bp, Artificial Sequence
  rs9564535_R
gcgttatccctcctgtgacatc

SEQ ID NO: 159 - DNA, 25 bp, Artificial Sequence
  rs1508494_F
ccctacattacccagggtagtctta

SEQ ID NO: 160 - DNA, 30 bp, Artificial Sequence
  rs1508494_R
ctaaacaaccataagtgttaaatgctgtga

SEQ ID NO: 161 - DNA, 22 bp, Artificial Sequence
  rs1876583_F
gggttttgcagatcacccaaga

SEQ ID NO: 162 - DNA, 22 bp, Artificial Sequence
  rs1876583_R
ccacccaatgaggctatgagtg

SEQ ID NO: 163 - DNA, 26 bp, Artificial Sequence
  rs9564626_F
tggaagaaatgtttgtcttctcagca

SEQ ID NO: 164 - DNA, 22 bp, Artificial Sequence
  rs9564626_R
gcagtcactggtgcttggttat

SEQ ID NO: 165 - DNA, 26 bp, Artificial Sequence
  rs9542105_F
gtcagccagaccaaaaacccatagga

SEQ ID NO: 166 - DNA, 29 bp, Artificial Sequence
  rs9542105_R
aacagttactataacaggtgggttacaag

SEQ ID NO: 167 - DNA, 28 bp, Artificial Sequence
  rs9592665_F
gccataatccaataaacttacgacccaa

SEQ ID NO: 168 - DNA, 25 bp, Artificial Sequence
  rs9592665_R
tgggagaggaagtcagagaatagac

SEQ ID NO: 169 - DNA, 27 bp, Artificial Sequence
  rs6562599_F
cttcaacaacaccataaaccaactagg

SEQ ID NO: 170 - DNA, 30 bp, Artificial Sequence
  rs6562599_R
gcatcccataacttttgttatactgtgttt

SEQ ID NO: 171 - DNA, 30 bp, Artificial Sequence
  rs9572308_F
ttttccacgttaacaaagaaaaagaagagg

SEQ ID NO: 172 - DNA, 30 bp, Artificial Sequence
  rs9572308_R
agcagggttaatcataatagtcaaaagagg

SEQ ID NO: 173 - DNA, 26 bp, Artificial Sequence
  rs7990298_F
cataaactgatagacccatgggtcaa

SEQ ID NO: 174 - DNA, 28 bp, Artificial Sequence
  rs7990298_R
tcaggtttctatctcttgcatttgtagg

SEQ ID NO: 175 - DNA, 25 bp, Artificial Sequence
  rs9542852_F
ggaatggtacccaaacctcatatgt

SEQ ID NO: 176 - DNA, 24 bp, Artificial Sequence
  rs9542852_R
gttttgtctgagtctctgctccat

SEQ ID NO: 177 - DNA, 30 bp, Artificial Sequence
  rs12586094_F
agatagcagtatactaaaggaaggaaagca

SEQ ID NO: 178 - DNA, 22 bp, Artificial Sequence
  rs12586094_R
tctccttaggcatctgacctgt

SEQ ID NO: 179 - DNA, 30 bp, Artificial Sequence
  rs9543171_F
catttctcagttttgtttgcatctctctag

SEQ ID NO: 180 - DNA, 30 bp, Artificial Sequence
  rs9543171_R
ggacagacagaagatattttaaactttgcc

SEQ ID NO: 181 - DNA, 22 bp, Artificial Sequence
  rs2031446_F
gtgttctctgcatcacctggaa

SEQ ID NO: 182 - DNA, 24 bp, Artificial Sequence
  rs2031446_R
ttgaagcagcacaggagacactga

SEQ ID NO: 183 - DNA, 25 bp, Artificial Sequence
  rs9544845_F
gctcacttttgatcaaagggagaca

SEQ ID NO: 184 - DNA, 22 bp, Artificial Sequence
  rs9544845_R
cttcaagaccctgtgtgatgct

SEQ ID NO: 185 - DNA, 30 bp, Artificial Sequence
  rs9574740_F
cacatacacagatacttacagaagcatacc

SEQ ID NO: 186 - DNA, 22 bp, Artificial Sequence
  rs9574740_R
catttgaaccagcgctgatgta

SEQ ID NO: 187 - DNA, 24 bp, Artificial Sequence
  rs7988209_F
agacctagaaaacacagttgccaa

SEQ ID NO: 188 - DNA, 29 bp, Artificial Sequence
  rs7988209_R
aaaattagtccctgtactctaagcttgtg

SEQ ID NO: 189 - DNA, 29 bp, Artificial Sequence
  rs9575364_F
agatttggtactccagaaatgaagtgaaa

SEQ ID NO: 190 - DNA, 23 bp, Artificial Sequence
  rs9575364_R
gcagacgaggtctggatcagtta

SEQ ID NO: 191 - DNA, 23 bp, Artificial Sequence
  rs7323558_F
cccatgccctagtcacctttaaa

SEQ ID NO: 192 - DNA, 30 bp, Artificial Sequence
  rs7323558_R
ccacatgcaatcacacagttatttatatct

SEQ ID NO: 193 - DNA, 23 bp, Artificial Sequence
  rs7325529_F
gaggcttagatgaagatgccctt

SEQ ID NO: 194 - DNA, 30 bp, Artificial Sequence
  rs7325529_R
gattacgctacttcatgcttagagatatgt

SEQ ID NO: 195 - DNA, 22 bp, Artificial Sequence
  rs4796869_F
gggcgagacttgcaacttttaa

SEQ ID NO: 196 - DNA, 27 bp, Artificial Sequence
  rs4796869_R
cacattcaaaacaggaataacgtgaca

SEQ ID NO: 197 - DNA, 26 bp, Artificial Sequence
  rs7236653_F
tttctgattgtactcgttgtttggga

SEQ ID NO: 198 - DNA, 27 bp, Artificial Sequence
  rs7236653_R
tattgggtgctatcaagagacagacat

SEQ ID NO: 199 - DNA, 23 bp, Artificial Sequence
  rs8095071_F
tgttgcaggagatgtgtgaagac

SEQ ID NO: 200 - DNA, 22 bp, Artificial Sequence
  rs8095071_R
acacacactgagctctgaagtg

SEQ ID NO: 201 - DNA, 30 bp, Artificial Sequence
  rs1787013_F
acaactcaacatttacgactgcttattaga

SEQ ID NO: 202 - DNA, 29 bp, Artificial Sequence
  rs1787013_R
tgtgatcgatttccaagttgtttgatttc

SEQ ID NO: 203 - DNA, 22 bp, Artificial Sequence
  rs9952107_F
gcagccagtgacccttgtaatc

SEQ ID NO: 204 - DNA, 28 bp, Artificial Sequence
  rs9952107_R
tccatctgtttattcaacaaacagtcct

SEQ ID NO: 205 - DNA, 28 bp, Artificial Sequence
  rs7231366_F
tgtttaatgatgtgttcaccatcagagt

SEQ ID NO: 206 - DNA, 29 bp, Artificial Sequence
  rs7231366_R
catatttgccaaaacctggaagtaactac

SEQ ID NO: 207 - DNA, 22 bp, Artificial Sequence
  rs7237577_F
acagagactgtgagctgtaggt

SEQ ID NO: 208 - DNA, 22 bp, Artificial Sequence
  rs7237577_R
tgcgctgtaataccacacatct

SEQ ID NO: 209 - DNA, 22 bp, Artificial Sequence
  rs11080646_F
gagacagggaaaggtgaacgta

SEQ ID NO: 210 - DNA, 30 bp, Artificial Sequence
  rs11080646_R
gcgcactacaaatattgacaattatcactt

SEQ ID NO: 211 - DNA, 26 bp, Artificial Sequence
  rs1284419_F
cccagatcaaataaccacatagacca

SEQ ID NO: 212 - DNA, 23 bp, Artificial Sequence
  rs1284419_R
atcaccctttaaagggtggttct

SEQ ID NO: 213 - DNA, 26 bp, Artificial Sequence
  rs9303869_F
actgctgagatgtcagttctacattc

SEQ ID NO: 214 - DNA, 27 bp, Artificial Sequence
  rs9303869_R
cggcctgaaatcttgaaataaatgaaa

SEQ ID NO: 215 - DNA, 22 bp, Artificial Sequence
  rs2487257_F
ctgtgctcagaactggtggata

SEQ ID NO: 216 - DNA, 22 bp, Artificial Sequence
  rs2487257_R
cctgtggactctttattcggca

SEQ ID NO: 217 - DNA, 22 bp, Artificial Sequence
  rs786018_F
catccacgtcaccacagagtaa

SEQ ID NO: 218 - DNA, 22 bp, Artificial Sequence
  rs786018_R
gtacactccaagtcctcaacga

SEQ ID NO: 219 - DNA, 23 bp, Artificial Sequence
  rs1030133_F
gacacaactgcagctgatgaaat

SEQ ID NO: 220 - DNA, 23 bp, Artificial Sequence
  rs1030133_R
tccaactttggtggaaggtgaaa

SEQ ID NO: 221 - DNA, 29 bp, Artificial Sequence
  rs968906_F
cggccaaaaacatgtaatacaaatacttg

SEQ ID NO: 222 - DNA, 30 bp, Artificial Sequence
  rs968906_R
acaagaaaaatggaagtgaaacaacaaaga

SEQ ID NO: 223 - DNA, 23 bp, Artificial Sequence
  rs10210_F
gggataaagaaggcgagttctga

SEQ ID NO: 224 - DNA, 22 bp, Artificial Sequence
  rs10210_R
gggcacgtgggaatgttaagaa

SEQ ID NO: 225 - DNA, 25 bp, Artificial Sequence
  rs892430_F
catttatgaggtgagcccatgaagt

SEQ ID NO: 226 - DNA, 23 bp, Artificial Sequence
  rs892430_R
gtggaggaggtcaaagggcaaaa

SEQ ID NO: 227 - DNA, 23 bp, Artificial Sequence
  rs12327010_F
acactgaagacccttctctctct

SEQ ID NO: 228 - DNA, 22 bp, Artificial Sequence
  rs12327010_R
atagtgtgcctaccacccacta

SEQ ID NO: 229 - DNA, 25 bp, Artificial Sequence
  rs716510_F
ccctccttactcctcctctatactt

SEQ ID NO: 230 - DNA, 30 bp, Artificial Sequence
  rs716510_R
gaaatctactgaactcgtttgttcacaaaa

SEQ ID NO: 231 - DNA, 30 bp, Artificial Sequence
  rs6508351_F
aaaaagatacgatccagaatgatttggaaa

SEQ ID NO: 232 - DNA, 30 bp, Artificial Sequence
  rs6508351_R
gtgtcatttgtttcttcgatgtttgataca

SEQ ID NO: 233 - DNA, 23 bp, Artificial Sequence
  rs7228099_F
gactcatacctgttcactgtgca

SEQ ID NO: 234 - DNA, 30 bp, Artificial Sequence
  rs7228099_R
gaaacaaagtaagaactgctaagagaaagg

SEQ ID NO: 235 - DNA, 20 bp, Artificial Sequence
  rs12961741_F
atgagagaccacccactgct

SEQ ID NO: 236 - DNA, 33 bp, Artificial Sequence
  rs12961741_R
atttgttcaaagttataaaggtacaggatatca

SEQ ID NO: 237 - DNA, 29 bp, Artificial Sequence
  rs1834545_F
atagcatgattccaatttgtgaaaaacgt

SEQ ID NO: 238 - DNA, 24 bp, Artificial Sequence
  rs1834545_R
gcagataaaagtgcccttgagagt

SEQ ID NO: 239 - DNA, 27 bp, Artificial Sequence
  rs17533_F
aaggaaaactagtaggaggtacttcca

SEQ ID NO: 240 - DNA, 22 bp, Artificial Sequence
  rs17533_R
gggtgcccacatccttatcatc

SEQ ID NO: 241 - DNA, 24 bp, Artificial Sequence
  rs273696_F
gaaaggtgctattagcctgggaaa

SEQ ID NO: 242 - DNA, 30 bp, Artificial Sequence
  rs273696_R
ggaatgagcatgtgttacttttacaattca

SEQ ID NO: 243 - DNA, 24 bp, Artificial Sequence
  rs273701_F
tcctcatccttcttgccctgatta

SEQ ID NO: 244 - DNA, 22 bp, Artificial Sequence
  rs273701_R
gcaggtgtaggtggagcatatt

SEQ ID NO: 245 - DNA, 29 bp, Artificial Sequence
  rs9303900_F
attcctactgggaaataccttaagaacaa

SEQ ID NO: 246 - DNA, 28 bp, Artificial Sequence
  rs9303900_R
cttacgcttgtaattttactctttggga

SEQ ID NO: 247 - DNA, 26 bp, Artificial Sequence
  rs1785745_F
cccatcgatcatatcatcttgtctct

SEQ ID NO: 248 - DNA, 29 bp, Artificial Sequence
  rs1785745_R
aattcattcagtgtgctaaattctgaagc

SEQ ID NO: 249 - DNA, 22 bp, Artificial Sequence
  rs1785739_F
ggactccaaccctccctctaaa

SEQ ID NO: 250 - DNA, 22 bp, Artificial Sequence
  rs1785739_R
catgcacaatgtgcattgccta

SEQ ID NO: 251 - DNA, 29 bp, Artificial Sequence
  rs8096605_F
gttccatttttagtaaggctttccatgtt

SEQ ID NO: 252 - DNA, 23 bp, Artificial Sequence
  rs8096605_R
gggaaccacactgagctcctata

SEQ ID NO: 253 - DNA, 26 bp, Artificial Sequence
  rs1786648_F
gtagtcatagctactctgaggtggaa

SEQ ID NO: 254 - DNA, 26 bp, Artificial Sequence
  rs1786648_R
gaaaatcttagcccagtgcaagtaag

SEQ ID NO: 255 - DNA, 29 bp, Artificial Sequence
  rs8096542_F
gaatgatagaaaatgttcccaaaaagcca

SEQ ID NO: 256 - DNA, 28 bp, Artificial Sequence
  rs8096542_R
tatccttttgctgtaatttgtgtctggt

SEQ ID NO: 257 - DNA, 27 bp, Artificial Sequence
  rs4800786_F
attgcaattatcagcttgcataatggg

SEQ ID NO: 258 - DNA, 30 bp, Artificial Sequence
  rs4800786_R
gacaatgtaggtgttgattctgattctact

SEQ ID NO: 259 - DNA, 30 bp, Artificial Sequence
  rs16943649_F
actaaaaggtctatgagaaggtctctacac

SEQ ID NO: 260 - DNA, 29 bp, Artificial Sequence
  rs16943649_R
tcttccaatctgtgaaacaattatctgct

SEQ ID NO: 261 - DNA, 22 bp, Artificial Sequence
  rs939139_F
aggcacgtgaagtttgcatttg

SEQ ID NO: 262 - DNA, 25 bp, Artificial Sequence
  rs939139_R
ggcagaaagcagatgttattaaggc

SEQ ID NO: 263 - DNA, 30 bp, Artificial Sequence
  rs6508502_F
agttgtcaaatcaaaggataaatgtgtgtg

SEQ ID NO: 264 - DNA, 29 bp, Artificial Sequence
  rs6508502_R
gatttgaccaatgtttctcctgaggataa

SEQ ID NO: 265 - DNA, 22 bp, Artificial Sequence
  rs11083386_F
ccatgccaccctctgaagaatg

SEQ ID NO: 266 - DNA, 30 bp, Artificial Sequence
  rs11083386_R
ttttaaatgtccatgagaacacaggataca

SEQ ID NO: 267 - DNA, 28 bp, Artificial Sequence
  rs2419027_F
gtgggacaaaaatcaattgcatagaact

SEQ ID NO: 268 - DNA, 22 bp, Artificial Sequence
  rs2419027_R
tcccatttcactcccacaggta

SEQ ID NO: 269 - DNA, 29 bp, Artificial Sequence
  rs2419041_F
ctgaacaatgaaaacttcactttctcaca

SEQ ID NO: 270 - DNA, 30 bp, Artificial Sequence
  rs2419041_R
tgtgcatgaatttcagaaaaataggcatta

SEQ ID NO: 271 - DNA, 30 bp, Artificial Sequence
  rs2729429_F
tttttggcaatgagtatatattgggctagt

SEQ ID NO: 272 - DNA, 28 bp, Artificial Sequence
  rs2729429_R
catgagaactctatcgtaagaactgcaa

SEQ ID NO: 273 - DNA, 28 bp, Artificial Sequence
  rs17711702_F
agcagatgatagtagaagaacacagaga

SEQ ID NO: 274 - DNA, 30 bp, Artificial Sequence
  rs17711702_R
tctgacatgtctctcatatgcaagtctata

SEQ ID NO: 275 - DNA, 22 bp, Artificial Sequence
  rs9957591_F
ggactccaaccctccctctaaa

SEQ ID NO: 276 - DNA, 22 bp, Artificial Sequence
  rs9957591_R
catgcacaatgtgcattgccta

SEQ ID NO: 277 - DNA, 23 bp, Artificial Sequence
  rs1026123_F
cccagaaggcattaggaaaacca

SEQ ID NO: 278 - DNA, 30 bp, Artificial Sequence
  rs1026123_R
gggttttgagccttacagattttattttga

SEQ ID NO: 279 - DNA, 25 bp, Artificial Sequence
  rs336279_F
gtctgttcacatcttgagtatggca

SEQ ID NO: 280 - DNA, 30 bp, Artificial Sequence
  rs336279_R
ctgccttgtagagtagctgtaatatttaca

SEQ ID NO: 281 - DNA, 26 bp, Artificial Sequence
  rs9949868_F
ggaatccaggtccataaagagaaagg

SEQ ID NO: 282 - DNA, 28 bp, Artificial Sequence
  rs9949868_R
gccactcaattcatattgttgagctgta

SEQ ID NO: 283 - DNA, 30 bp, Artificial Sequence
  rs8097023_F
ggatctaaaaattcaatggtgcctacatac

SEQ ID NO: 284 - DNA, 25 bp, Artificial Sequence
  rs8097023_R
tagtatccagttggaagggatggaa

SEQ ID NO: 285 - DNA, 27 bp, Artificial Sequence
  rs11081004_F
gaaacttactatgtgagtgaggaggtt

SEQ ID NO: 286 - DNA, 27 bp, Artificial Sequence
  rs11081004_R
tgaattccacaatgaccttaaaagggt

SEQ ID NO: 287 - DNA, 22 bp, Artificial Sequence
  rs878971_F
ccacccacacaggtgattctaa

SEQ ID NO: 288 - DNA, 23 bp, Artificial Sequence
  rs878971_R
ccaggaagctgcatttgacagat

SEQ ID NO: 289 - DNA, 29 bp, Artificial Sequence
  rs1662304_F
gcctaatttatttcaccagttacacaagg

SEQ ID NO: 290 - DNA, 30 bp, Artificial Sequence
  rs1662304_R
caagtattagctatacatggcaaaaagcaa

SEQ ID NO: 291 - DNA, 23 bp, Artificial Sequence
  rs11081037_F
agttactgagacagctcccagaa

SEQ ID NO: 292 - DNA, 30 bp, Artificial Sequence
  rs11081037_R
aaagagaaacaaaagagtttcagagaggaa

SEQ ID NO: 293 - DNA, 25 bp, Artificial Sequence
  rs1790649_F
gtggccagatccatgaatataaggt

SEQ ID NO: 294 - DNA, 29 bp, Artificial Sequence
  rs1790649_R
agaaagaaagaaatagtgtgtgaagtgct

SEQ ID NO: 295 - DNA, 22 bp, Artificial Sequence
  rs2090036_F
ggcacttggcccatctctattt

SEQ ID NO: 296 - DNA, 23 bp, Artificial Sequence
  rs2090036_R
tatacctgctcacctcacccatt

SEQ ID NO: 297 - DNA, 28 bp, Artificial Sequence
  rs8097433_F
attagttctctggttggtccacatttag

SEQ ID NO: 298 - DNA, 22 bp, Artificial Sequence
  rs8097433_R
ccagtggaaataggccaaggaa

SEQ ID NO: 299 - DNA, 28 bp, Artificial Sequence
  rs1390431_F
gctgaaatccaggacataataagtaggt

SEQ ID NO: 300 - DNA, 27 bp, Artificial Sequence
  rs1390431_R
tgctgaatttctttcatcacaacttgg

SEQ ID NO: 301 - DNA, 22 bp, Artificial Sequence
  rs12967616_F
tggcacccagtgatttaagtgg

SEQ ID NO: 302 - DNA, 24 bp, Artificial Sequence
  rs12967616_R
gcccagcccaacatttcttttaat

SEQ ID NO: 303 - DNA, 25 bp, Artificial Sequence
  rs12966492_F
tgtgacttctagttaggcaaggagt

SEQ ID NO: 304 - DNA, 30 bp, Artificial Sequence
  rs12966492_R
tcatgatttgtgttttcaagaagaatgtgg

SEQ ID NO: 305 - DNA, 23 bp, Artificial Sequence
  rs6507196_F
tcgccagcctgctaatttttaga

SEQ ID NO: 306 - DNA, 26 bp, Artificial Sequence
  rs6507196_R
cccgagacccatcaattctatctact

SEQ ID NO: 307 - DNA, 30 bp, Artificial Sequence
  rs4799910_F
tgttattgctactttacagaggaggaaatg

SEQ ID NO: 308 - DNA, 23 bp, Artificial Sequence
  rs4799910_R
gggatgcaccaatctcacactac

SEQ ID NO: 309 - DNA, 23 bp, Artificial Sequence
  rs1893455_F
gaccctcattggtttgaaaggga

SEQ ID NO: 310 - DNA, 22 bp, Artificial Sequence
  rs1893455_R
ggagattctgctccctggtttg

SEQ ID NO: 311 - DNA, 22 bp, Artificial Sequence
  rs9947829_F
gccgaagcaaattctgcacttg

SEQ ID NO: 312 - DNA, 30 bp, Artificial Sequence
  rs9947829_R
cttcatttgaaatgaaccttgagctatgag

SEQ ID NO: 313 - DNA, 28 bp, Artificial Sequence
  rs8090831_F
atttctttgtgataatggccagatggta

SEQ ID NO: 314 - DNA, 22 bp, Artificial Sequence
  rs8090831_R
cctccttgtgcttgtaccatga

SEQ ID NO: 315 - DNA, 22 bp, Artificial Sequence
  rs9967142_F
actggagttcacacagcctcta

SEQ ID NO: 316 - DNA, 23 bp, Artificial Sequence
  rs9967142_R
taagtgaggccatccaggatcat

SEQ ID NO: 317 - DNA, 30 bp, Artificial Sequence
  rs1567612_F
tatccaaatacatactttgtccaaagtgca

SEQ ID NO: 318 - DNA, 29 bp, Artificial Sequence
  rs1567612_R
tgaggtagtgttcttggagtgatataact

SEQ ID NO: 319 - DNA, 25 bp, Artificial Sequence
  rs1145560_F
aaaaactgcttgagagtgtaggtga

SEQ ID NO: 320 - DNA, 28 bp, Artificial Sequence
  rs1145560_R
gagaattctgagttcctagttttgtgtg

SEQ ID NO: 321 - DNA, 26 bp, Artificial Sequence
  rs991045_F
cctgtttaatctccaagaagcaatgc

SEQ ID NO: 322 - DNA, 25 bp, Artificial Sequence
  rs991045_R
gcaagtcactgaattttcttcagct

SEQ ID NO: 323 - DNA, 29 bp, Artificial Sequence
  rs1593579_F
gacttatttacagctgttcctattaccca

SEQ ID NO: 324 - DNA, 30 bp, Artificial Sequence
  rs1593579_R
cctaatcatcttctaaaggtccacctctta

SEQ ID NO: 325 - DNA, 22 bp, Artificial Sequence
  rs4133291_F
gggattatcacccgcatgacta

SEQ ID NO: 326 - DNA, 26 bp, Artificial Sequence
  rs4133291_R
caggagacttactctatgcaaattgc

SEQ ID NO: 327 - DNA, 23 bp, Artificial Sequence
  rs9945284_F
ggccattttcagcctctcttctc

SEQ ID NO: 328 - DNA, 22 bp, Artificial Sequence
  rs9945284_R
cgcctggccatgttacatgtat

SEQ ID NO: 329 - DNA, 30 bp, Artificial Sequence
  rs9967440_F
tgatactatctggtcatctgtctttagtca

SEQ ID NO: 330 - DNA, 30 bp, Artificial Sequence
  rs9967440_R
actctgggatcataagatagctattccaaa

SEQ ID NO: 331 - DNA, 27 bp, Artificial Sequence
  rs6506138_F
caagatttactgtagcaacttgcacat

SEQ ID NO: 332 - DNA, 22 bp, Artificial Sequence
  rs6506138_R
cggagatgtgttcctccatgat

SEQ ID NO: 333 - DNA, 30 bp, Artificial Sequence
  rs11872146_F
acctacaaaaatgtgtttgaagtatcatgc

SEQ ID NO: 334 - DNA, 23 bp, Artificial Sequence
  rs11872146_R
atcactaaaggctgtcatggcaa

SEQ ID NO: 335 - DNA, 30 bp, Artificial Sequence
  rs4407150_F
tggtatgtagagaacattatctgtgtgtga

SEQ ID NO: 336 - DNA, 30 bp, Artificial Sequence
  rs4407150_R
aaacaactgcaaagtatcttaagctgattt

SEQ ID NO: 337 - DNA, 28 bp, Artificial Sequence
  rs2222370_F
cccaaatgattagtaagtgggtgaaaaa

SEQ ID NO: 338 - DNA, 30 bp, Artificial Sequence
  rs2222370_R
gtctggcaagagttttctcaattttgttta

SEQ ID NO: 339 - DNA, 24 bp, Artificial Sequence
  rs2320747_F
ccttctctagcagaaaattccgca

SEQ ID NO: 340 - DNA, 22 bp, Artificial Sequence
  rs2320747_R
acccgacattcagcgaattgaa

SEQ ID NO: 341 - DNA, 29 bp, Artificial Sequence
  rs4798412_F
gaagacctgagtttctagaagaattgagt

SEQ ID NO: 342 - DNA, 22 bp, Artificial Sequence
  rs4798412_R
actggagtaggatgagcctcat

SEQ ID NO: 343 - DNA, 28 bp, Artificial Sequence
  rs6506332_F
ctgcaaaatgcaacaacatacatgaaac

SEQ ID NO: 344 - DNA, 28 bp, Artificial Sequence
  rs6506332_R
aagccttttagtttggtcaaaataagcc

SEQ ID NO: 345 - DNA, 29 bp, Artificial Sequence
  rs694182_F
tgcttttaaaggatggtatttctggatga

SEQ ID NO: 346 - DNA, 26 bp, Artificial Sequence
  rs694182_R
tgagattcagcttatgcattgtgaga

SEQ ID NO: 347 - DNA, 25 bp, Artificial Sequence
  rs341237_F
cacccttcaaacacaatctgacatt

SEQ ID NO: 348 - DNA, 23 bp, Artificial Sequence
  rs341237_R
gatgagggtgtgattggattcca

SEQ ID NO: 349 - DNA, 28 bp, Artificial Sequence
  rs9958938_F
tctttggtactttctttcattgtccact

SEQ ID NO: 350 - DNA, 23 bp, Artificial Sequence
  rs9958938_R
gctgagcatatgttccatgcaag

SEQ ID NO: 351 - DNA, 27 bp, Artificial Sequence
  rs4798479_F
cccttgtctagcttgaatattgaactg

SEQ ID NO: 352 - DNA, 28 bp, Artificial Sequence
  rs4798479_R
gctagatggaagtatccaatgggaaaaa

SEQ ID NO: 353 - DNA, 30 bp, Artificial Sequence
  rs10502290_F
acattttgtgatctatttttcaagaagcgt

SEQ ID NO: 354 - DNA, 29 bp, Artificial Sequence
  rs10502290_R
ctgaaaggaacctcatttggtgattttat

SEQ ID NO: 355 - DNA, 22 bp, Artificial Sequence
  rs2298583_F
gtgagtcagctaccagacccta

SEQ ID NO: 356 - DNA, 30 bp, Artificial Sequence
  rs2298583_R
gccctactcttggtattaaatagaaacgat

SEQ ID NO: 357 - DNA, 22 bp, Artificial Sequence
  rs3786355_F
ccagtatcctccggtctttgtt

SEQ ID NO: 358 - DNA, 23 bp, Artificial Sequence
  rs3786355_R
acgactgtacattcatgtggctt

SEQ ID NO: 359 - DNA, 23 bp, Artificial Sequence
  rs12185460_F
cactaaagggtgtagtggaaggt

SEQ ID NO: 360 - DNA, 28 bp, Artificial Sequence
  rs12185460_R
tctctatatgactgcctatgtggagatg

SEQ ID NO: 361 - DNA, 22 bp, Artificial Sequence
  rs16951141_F
agggaagtcttccctgaaggta

SEQ ID NO: 362 - DNA, 22 bp, Artificial Sequence
  rs16951141_R
ccctgtaagacccgttgctttt

SEQ ID NO: 363 - DNA, 30 bp, Artificial Sequence
  rs7234111_F
gcactgtgattccattaataagaagtcatc

SEQ ID NO: 364 - DNA, 22 bp, Artificial Sequence
  rs7234111_R
ggtgggaaaggcctggaaatta

SEQ ID NO: 365 - DNA, 27 bp, Artificial Sequence
  rs16951664_F
cgccatgcttcttgtatagtctgtaaa

SEQ ID NO: 366 - DNA, 22 bp, Artificial Sequence
  rs16951664_R
ccatcgacgcatctttgttgtg

SEQ ID NO: 367 - DNA, 23 bp, Artificial Sequence
  rs602212_F
tgaagctgtgatctaccggtcta

SEQ ID NO: 368 - DNA, 29 bp, Artificial Sequence
  rs602212_R
gttctcacttccacaagctatatactctc

SEQ ID NO: 369 - DNA, 23 bp, Artificial Sequence
  rs8092926_F
gaaagacaagcccagactctgaa

SEQ ID NO: 370 - DNA, 22 bp, Artificial Sequence
  rs8092926_R
gctcagaacagctccaggtatt

SEQ ID NO: 371 - DNA, 22 bp, Artificial Sequence
  rs11660737_F
ggtctccttcccagaacctact

SEQ ID NO: 372 - DNA, 22 bp, Artificial Sequence
  rs11660737_R
ccaacagagggccaaagaattc

SEQ ID NO: 373 - DNA, 23 bp, Artificial Sequence
  rs4239334_F
cgcttctgttcatcaacttcctg

SEQ ID NO: 374 - DNA, 24 bp, Artificial Sequence
  rs4239334_R
aagcacagtgtctgtttcactaca

SEQ ID NO: 375 - DNA, 23 bp, Artificial Sequence
  rs8095514_F
acccaggcatctgtggtttttag

SEQ ID NO: 376 - DNA, 30 bp, Artificial Sequence
  rs8095514_R
gttattgtcctttacaacttctgtagtcct

SEQ ID NO: 377 - DNA, 26 bp, Artificial Sequence
  rs7235989_F
ggagaaagtaaaacatgctgccaatt

SEQ ID NO: 378 - DNA, 27 bp, Artificial Sequence
  rs7235989_R
tttttcactttcagcacagtattgcaa

SEQ ID NO: 379 - DNA, 24 bp, Artificial Sequence
  rs12606001_F
gccttcctggtcatatcgttttcc

SEQ ID NO: 380 - DNA, 23 bp, Artificial Sequence
  rs12606001_R
aaaaggcttcagcaaaggtttcc

SEQ ID NO: 381 - DNA, 28 bp, Artificial Sequence
  rs2027667_F
ccactatgcctggcctctttatttaaaa

SEQ ID NO: 382 - DNA, 30 bp, Artificial Sequence
  rs2027667_R
atttctttcttctcatttccaatgctttcc

SEQ ID NO: 383 - DNA, 26 bp, Artificial Sequence
  rs329027_F
ccaaaaggacttagcatgctttcttt

SEQ ID NO: 384 - DNA, 24 bp, Artificial Sequence
  rs329027_R
ggagccagtctgtctggatttaac

SEQ ID NO: 385 - DNA, 26 bp, Artificial Sequence
  rs403093_F
cgggaggttaacatttcagaactaga

SEQ ID NO: 386 - DNA, 29 bp, Artificial Sequence
  rs403093_R
gtgacagataatagtagaaattggcggtt

SEQ ID NO: 387 - DNA, 27 bp, Artificial Sequence
  rs8087127_F
cccaggaatcagcattttaacaaactt

SEQ ID NO: 388 - DNA, 30 bp, Artificial Sequence
  rs8087127_R
aaaattgatcagaaccagagataaagagca

SEQ ID NO: 389 - DNA, 22 bp, Artificial Sequence
  rs1023868_F
gcctgctcatgatggactgata

SEQ ID NO: 390 - DNA, 24 bp, Artificial Sequence
  rs1023868_R
cacaggatgcagatgaagagactt

SEQ ID NO: 391 - DNA, 29 bp, Artificial Sequence
  rs2775537_F
ggtgtattgaagtcttctgtaataacaca

SEQ ID NO: 392 - DNA, 22 bp, Artificial Sequence
  rs2775537_R
gacggaattcaggctgagacaa

SEQ ID NO: 393 - DNA, 25 bp, Artificial Sequence
  rs2821796_F
gggaaaatgtgttagcaatgaactc

SEQ ID NO: 394 - DNA, 31 bp, Artificial Sequence
  rs2821796_R
aatgtgttggtgtaaacacaactttattttt

SEQ ID NO: 395 - DNA, 24 bp, Artificial Sequence
  rs8134986_F
agagactgccagaaaaacagagtc

SEQ ID NO: 396 - DNA, 23 bp, Artificial Sequence
  rs8134986_R
cctctgttcccgattcactgata

SEQ ID NO: 397 - DNA, 28 bp, Artificial Sequence
  rs12482146_F
atatgcctcatgtatttctttgttccca

SEQ ID NO: 398 - DNA, 23 bp, Artificial Sequence
  rs12482146_R
cctgttgtggattgcttttctgg

SEQ ID NO: 399 - DNA, 29 bp, Artificial Sequence
  rs431864_F
tgcaaaatttggctttttaaatcatctgc

SEQ ID NO: 400 - DNA, 24 bp, Artificial Sequence
  rs431864_R
ctttttggattcagcaaaggccat

SEQ ID NO: 401 - DNA, 25 bp, Artificial Sequence
  rs435081_F
tttatgtggatgtgccaagctgata

SEQ ID NO: 402 - DNA, 30 bp, Artificial Sequence
  rs435081_R
gattccagatgatgcacttaaccagtatat

SEQ ID NO: 403 - DNA, 30 bp, Artificial Sequence
  rs439146_F
tcctgattccttatcattcatggtaagtct

SEQ ID NO: 404 - DNA, 29 bp, Artificial Sequence
  rs439146_R
accaatgttgtacttaattacaggaagct

SEQ ID NO: 405 - DNA, 27 bp, Artificial Sequence
  rs416083_F
acacagacccaaaccctatcaatacta

SEQ ID NO: 406 - DNA, 30 bp, Artificial Sequence
  rs416083_R
ggagaagaggcaaattccaaaaatacttac

SEQ ID NO: 407 - DNA, 27 bp, Artificial Sequence
  rs375484_F
tcagaaagtgttgtgaacatgaagtca

SEQ ID NO: 408 - DNA, 26 bp, Artificial Sequence
  rs375484_R
gctctcaaccattttgccaaaaacaa

SEQ ID NO: 409 - DNA, 22 bp, Artificial Sequence
  rs377191_F
cacgcgttttgttcgagttcta

SEQ ID NO: 410 - DNA, 29 bp, Artificial Sequence
  rs377191_R
gggattatggaatttattgggcaaaaagg

SEQ ID NO: 411 - DNA, 26 bp, Artificial Sequence
  rs386838_F
tacaaatccctgggaatatgcttagc

SEQ ID NO: 412 - DNA, 30 bp, Artificial Sequence
  rs386838_R
agaatgtgtcattcagagaaaagaatgagt

SEQ ID NO: 413 - DNA, 27 bp, Artificial Sequence
  rs4588273_F
agaacagtgttcagcaaaatacagtca

SEQ ID NO: 414 - DNA, 26 bp, Artificial Sequence
  rs4588273_R
ctaccaggtcacagaggaggagacaa

SEQ ID NO: 415 - DNA, 23 bp, Artificial Sequence
  rs2013669_F
ctacgcccagccaagaaatgaga

SEQ ID NO: 416 - DNA, 30 bp, Artificial Sequence
  rs2013669_R
cgtctctttaagtattgtgaacctaaggaa

SEQ ID NO: 417 - DNA, 29 bp, Artificial Sequence
  rs2822618_F
agttatcatgtgatgatcccatgtttgat

SEQ ID NO: 418 - DNA, 30 bp, Artificial Sequence
  rs2822618_R
cagacaagcaaagaagaagagtgtttaatt

SEQ ID NO: 419 - DNA, 23 bp, Artificial Sequence
  rs2822648_F
ctctcctcgtatctcacctcctt

SEQ ID NO: 420 - DNA, 23 bp, Artificial Sequence
  rs2822648_R
gggcatctttcttcgcttatgtt

SEQ ID NO: 421 - DNA, 22 bp, Artificial Sequence
  rs2242661_F
gcaaggacttcatctaccgtga

SEQ ID NO: 422 - DNA, 26 bp, Artificial Sequence
  rs2242661_R
acgcgctttaaaaggaattaaaacga

SEQ ID NO: 423 - DNA, 30 bp, Artificial Sequence
  rs2822661_F
ctatgctggaattagagtacaagattcagg

SEQ ID NO: 424 - DNA, 30 bp, Artificial Sequence
  rs2822661_R
tgcctaaagcgagatgatttataaagaact

SEQ ID NO: 425 - DNA, 24 bp, Artificial Sequence
  rs11088040_F
tggcatccaggatatttgcatctt

SEQ ID NO: 426 - DNA, 30 bp, Artificial Sequence
  rs11088040_R
aagtttaccaaataaaggaggtttgtgaga

SEQ ID NO: 427 - DNA, 30 bp, Artificial Sequence
  rs760345_F
ttttactttgcaaaagtgacattgtcttca

SEQ ID NO: 428 - DNA, 30 bp, Artificial Sequence
  rs760345_R
ctatgacaaaaagcgtagaaagacatttga

SEQ ID NO: 429 - DNA, 29 bp, Artificial Sequence
  rs467140_F
gatgtatgtgaatatcctggaaatttgcc

SEQ ID NO: 430 - DNA, 27 bp, Artificial Sequence
  rs467140_R
ttaaaaagcatgtcgtctgattttggg

SEQ ID NO: 431 - DNA, 23 bp, Artificial Sequence
  rs461853_F
cttctcctcagtcacaaggagtt

SEQ ID NO: 432 - DNA, 23 bp, Artificial Sequence
  rs461853_R
ccgtttcgtggaaagaagaaagt

SEQ ID NO: 433 - DNA, 30 bp, Artificial Sequence
  rs2822809_F
ctaaagagaagatttggtgtgtgaaatgtt

SEQ ID NO: 434 - DNA, 23 bp, Artificial Sequence
  rs2822809_R
ctcctgccacaaagcaacaaaac

SEQ ID NO: 435 - DNA, 28 bp, Artificial Sequence
  rs4817371_F
tcgttttccttcattaacatagggtcaa

SEQ ID NO: 436 - DNA, 29 bp, Artificial Sequence
  rs4817371_R
ggtaaagagcacgcttattttatcagtct

SEQ ID NO: 437 - DNA, 23 bp, Artificial Sequence
  rs1018676_F
tgagcatccaccatcagtgagta

SEQ ID NO: 438 - DNA, 30 bp, Artificial Sequence
  rs1018676_R
ctattcattgggtattctactccatttggt

SEQ ID NO: 439 - DNA, 27 bp, Artificial Sequence
  rs926130_F
ttcccttcccagtactaccatatcatt

SEQ ID NO: 440 - DNA, 29 bp, Artificial Sequence
  rs926130_R
cctgtgagcaaaaggatctataacaagag

SEQ ID NO: 441 - DNA, 28 bp, Artificial Sequence
  rs2822965_F
aagaggatgtgagggtatctttttaagc

SEQ ID NO: 442 - DNA, 29 bp, Artificial Sequence
  rs2822965_R
aaagaatgcatatggagagggtatagaga

SEQ ID NO: 443 - DNA, 29 bp, Artificial Sequence
  rs2822973_F
gtctacacaggatcaggattatcaagatg

SEQ ID NO: 444 - DNA, 24 bp, Artificial Sequence
  rs2822973_R
gtatttctccacgttccctcactt

SEQ ID NO: 445 - DNA, 24 bp, Artificial Sequence
  rs2822975_F
gtgtgtgtgtgcaagtaacagaag

SEQ ID NO: 446 - DNA, 29 bp, Artificial Sequence
  rs2822975_R
aacatcctagaaatggaaaattagccagt

SEQ ID NO: 447 - DNA, 30 bp, Artificial Sequence
  rs1002460_F
agtagttctgttcttggaatacttgttctg

SEQ ID NO: 448 - DNA, 28 bp, Artificial Sequence
  rs1002460_R
agtattctcagtgtggaattacattcgg

SEQ ID NO: 449 - DNA, 30 bp, Artificial Sequence
  rs2256417_F
tatagtctcgctactagatttttggtcact

SEQ ID NO: 450 - DNA, 22 bp, Artificial Sequence
  rs2256417_R
gttgctgacttggcatttggtt

SEQ ID NO: 451 - DNA, 28 bp, Artificial Sequence
  rs2245411_F
acttccacatttgaaagataagcttcca

SEQ ID NO: 452 - DNA, 30 bp, Artificial Sequence
  rs2245411_R
tgctgctattattgcctacctattgaaatt

SEQ ID NO: 453 - DNA, 28 bp, Artificial Sequence
  rs2736084_F
aaagagatatatactgggcactcactga

SEQ ID NO: 454 - DNA, 23 bp, Artificial Sequence
  rs2736084_R
cctccttcctttgctcattctga

SEQ ID NO: 455 - DNA, 25 bp, Artificial Sequence
  rs975336_F
ggcttgaatatggtcacatttccag

SEQ ID NO: 456 - DNA, 22 bp, Artificial Sequence
  rs975336_R
aaacacgcgattgctgttactg

SEQ ID NO: 457 - DNA, 23 bp, Artificial Sequence
  rs2823145_F
tttattggctgtagggccagttt

SEQ ID NO: 458 - DNA, 24 bp, Artificial Sequence
  rs2823145_R
cttaaggtccaagatgctggtttc

SEQ ID NO: 459 - DNA, 26 bp, Artificial Sequence
  rs2823152_F
tctttttaagacaggcttgctactgt

SEQ ID NO: 460 - DNA, 28 bp, Artificial Sequence
  rs2823152_R
aattagagtgagaatgttcaaccagctt

SEQ ID NO: 461 - DNA, 27 bp, Artificial Sequence
  rs2823169_F
tttctcttcccaccaagaataattccc

SEQ ID NO: 462 - DNA, 30 bp, Artificial Sequence
  rs2823169_R
cagagacatgcttcgtggattaattaacta

SEQ ID NO: 463 - DNA, 30 bp, Artificial Sequence
  rs413285_F
gtgaaaattggctaatcttagaaggaaagc

SEQ ID NO: 464 - DNA, 22 bp, Artificial Sequence
  rs413285_R
cccaaccggatcttaagctcat

SEQ ID NO: 465 - DNA, 24 bp, Artificial Sequence
  rs2205533_F
tggaagaaaacttctgggcaatca

SEQ ID NO: 466 - DNA, 25 bp, Artificial Sequence
  rs2205533_R
ggattccaccaaggtaaagagagga

SEQ ID NO: 467 - DNA, 23 bp, Artificial Sequence
  rs2823795_F
cagacttcctctgtgctgttcat

SEQ ID NO: 468 - DNA, 30 bp, Artificial Sequence
  rs2823795_R
cagcatgaactaaaatagatggaagttgat

SEQ ID NO: 469 - DNA, 27 bp, Artificial Sequence
  rs2823809_F
taggctatagcaataggcttaagaggt

SEQ ID NO: 470 - DNA, 25 bp, Artificial Sequence
  rs2823809_R
ctcattcatacacactcccagcatt

SEQ ID NO: 471 - DNA, 26 bp, Artificial Sequence
  rs2823983_F
gtgctacagttaagatggaagttggt

SEQ ID NO: 472 - DNA, 22 bp, Artificial Sequence
  rs2823983_R
gcatcctccaggagtaggagaa

SEQ ID NO: 473 - DNA, 24 bp, Artificial Sequence
  rs2824133_F
tcatacagacgcgagtgaaattga

SEQ ID NO: 474 - DNA, 27 bp, Artificial Sequence
  rs2824133_R
ccacttccaaattttggaaaacgagag

SEQ ID NO: 475 - DNA, 26 bp, Artificial Sequence
  rs10470201_F
gctctgagaaggacttccagtactta

SEQ ID NO: 476 - DNA, 29 bp, Artificial Sequence
  rs10470201_R
gcatatcaaagcataatacaccatgacca

SEQ ID NO: 477 - DNA, 23 bp, Artificial Sequence
  rs1014604_F
gccaaatccaaggctgaggaaat

SEQ ID NO: 478 - DNA, 30 bp, Artificial Sequence
  rs1014604_R
attacattgaaaagtctctatggcagtttg

SEQ ID NO: 479 - DNA, 26 bp, Artificial Sequence
  rs2223079_F
ctaagtggtttcaattacagcagcaa

SEQ ID NO: 480 - DNA, 29 bp, Artificial Sequence
  rs2223079_R
aaagtgagtgacagttgactaatactgtc

SEQ ID NO: 481 - DNA, 30 bp, Artificial Sequence
  rs9978408_F
ggaacttcacaatgcaatcacaaatattca

SEQ ID NO: 482 - DNA, 25 bp, Artificial Sequence
  rs9978408_R
ttggagcgtctgattattacgtgtt

SEQ ID NO: 483 - DNA, 28 bp, Artificial Sequence
  rs2226798_F
tgccatggcacatctttatctatgtaat

SEQ ID NO: 484 - DNA, 30 bp, Artificial Sequence
  rs2226798_R
aattaagtcctctgaaagacttccatttct

SEQ ID NO: 485 - DNA, 22 bp, Artificial Sequence
  rs2824238_F
ggtgaggagtcaagagagacca

SEQ ID NO: 486 - DNA, 30 bp, Artificial Sequence
  rs2824238_R
gcagccattgatgattatttcttagactca

SEQ ID NO: 487 - DNA, 29 bp, Artificial Sequence
  rs13046156_F
gttcagtgtttctgaaaatggataatgct

SEQ ID NO: 488 - DNA, 29 bp, Artificial Sequence
  rs13046156_R
ccaatgccatgctaattgtttttgtattt

SEQ ID NO: 489 - DNA, 29 bp, Artificial Sequence
  rs9984531_F
aaaaatgaagaagaactccaagaagttcg

SEQ ID NO: 490 - DNA, 30 bp, Artificial Sequence
  rs9984531_R
ggtaggttttcctttataggttactggatc

SEQ ID NO: 491 - DNA, 22 bp, Artificial Sequence
  rs1102617_F
atccagaaccaccactcaatcc

SEQ ID NO: 492 - DNA, 25 bp, Artificial Sequence
  rs1102617_R
cccagtgtctgttgtttttcctttg

SEQ ID NO: 493 - DNA, 29 bp, Artificial Sequence
  rs8127266_F
agcttcatattccatgtaatgaccatcac

SEQ ID NO: 494 - DNA, 28 bp, Artificial Sequence
  rs8127266_R
tctcaggcttaatattttgtgtgtgtgt

SEQ ID NO: 495 - DNA, 27 bp, Artificial Sequence
  rs208932_F
agtgacataaggacttatagcacctgt

SEQ ID NO: 496 - DNA, 22 bp, Artificial Sequence
  rs208932_R
ggctgcactagacagatgcaaa

SEQ ID NO: 497 - DNA, 30 bp, Artificial Sequence
  rs211962_F
gacggtcttacttttctttttcctttcttc

SEQ ID NO: 498 - DNA, 22 bp, Artificial Sequence
  rs211962_R
ccagggaatggcctcacctata

SEQ ID NO: 499 - DNA, 30 bp, Artificial Sequence
  rs2824376_F
aaattctccattaccagtgacagtttttac

SEQ ID NO: 500 - DNA, 30 bp, Artificial Sequence
  rs2824376_R
accagatgaaaaggaataaatcgcctataa

SEQ ID NO: 501 - DNA, 25 bp, Artificial Sequence
  rs243594_F
gggagattttccactgtaggtttct

SEQ ID NO: 502 - DNA, 26 bp, Artificial Sequence
  rs243594_R
ggatggtcagtacagtgtacagaatg

SEQ ID NO: 503 - DNA, 25 bp, Artificial Sequence
  rs243601_F
acccaaagacataaacttgcaagga

SEQ ID NO: 504 - DNA, 30 bp, Artificial Sequence
  rs243601_R
gctcctgtttagctttaactaactgacatt

SEQ ID NO: 505 - DNA, 29 bp, Artificial Sequence
  rs2183557_F
tgaaaatacagattgttagtgactggcat

SEQ ID NO: 506 - DNA, 30 bp, Artificial Sequence
  rs2183557_R
gagcttattagaatatctcaggcacatgaa

SEQ ID NO: 507 - DNA, 24 bp, Artificial Sequence
  rs1573442_F
aatatgaacaagggcagaagcact

SEQ ID NO: 508 - DNA, 26 bp, Artificial Sequence
  rs1573442_R
tgttctaataaggtggcctatgagga

SEQ ID NO: 509 - DNA, 23 bp, Artificial Sequence
  rs2824762_F
cttgaataaggcctggagctcaa

SEQ ID NO: 510 - DNA, 28 bp, Artificial Sequence
  rs2824762_R
caaaaattctaagcaaattggcattgca

SEQ ID NO: 511 - DNA, 25 bp, Artificial Sequence
  rs8132870_F
gaggactcatatctcaaatgcagct

SEQ ID NO: 512 - DNA, 29 bp, Artificial Sequence
  rs8132870_R
gatagagtattatgtttggctatgcccat

SEQ ID NO: 513 - DNA, 28 bp, Artificial Sequence
  rs2211938_F
atcaaacaggactgtgatgtcaaatagt

SEQ ID NO: 514 - DNA, 29 bp, Artificial Sequence
  rs2211938_R
gtttgatcacttaactacattttcgtgct

SEQ ID NO: 515 - DNA, 25 bp, Artificial Sequence
  rs2825516_F
agagggaactgtcatatattgctgc

SEQ ID NO: 516 - DNA, 30 bp, Artificial Sequence
  rs2825516_R
ccttggaccacatctacttactataaacct

SEQ ID NO: 517 - DNA, 25 bp, Artificial Sequence
  rs1491658_F
ccctttcacctttgcatgattttga

SEQ ID NO: 518 - DNA, 24 bp, Artificial Sequence
  rs1491658_R
gcaacgttcgtatcacatcaatgt

SEQ ID NO: 519 - DNA, 25 bp, Artificial Sequence
  rs863075_F
agttgcacagaagctgatcttatga

SEQ ID NO: 520 - DNA, 24 bp, Artificial Sequence
  rs863075_R
gttgaaccttgtgtaggttccctt

SEQ ID NO: 521 - DNA, 29 bp, Artificial Sequence
  rs1826318_F
ccagaaaatgatggtgaaagtaaaatcca

SEQ ID NO: 522 - DNA, 27 bp, Artificial Sequence
  rs1826318_R
tgtttcttgtagctgctttggtatgta

SEQ ID NO: 523 - DNA, 22 bp, Artificial Sequence
  rs7278137_F
gccttattgccagtagcagaca

SEQ ID NO: 524 - DNA, 27 bp, Artificial Sequence
  rs7278137_R
aaaattgagatttggtaaagctggcaa

SEQ ID NO: 525 - DNA, 25 bp, Artificial Sequence
  rs2825560_F
gacctgtgaagtctaagagttgact

SEQ ID NO: 526 - DNA, 30 bp, Artificial Sequence
  rs2825560_R
ccgtggtcccatgaaattataataccatat

SEQ ID NO: 527 - DNA, 30 bp, Artificial Sequence
  rs2825576_F
attgtgtagatcttacttaagatggttgga

SEQ ID NO: 528 - DNA, 29 bp, Artificial Sequence
  rs2825576_R
gggtaataggaggaattatggaaaacagt

SEQ ID NO: 529 - DNA, 29 bp, Artificial Sequence
  rs2825578_F
tcgactttgaattcagacacttctaaagt

SEQ ID NO: 530 - DNA, 30 bp, Artificial Sequence
  rs2825578_R
cagaaattaatagccagcagtagtgatttt

SEQ ID NO: 531 - DNA, 25 bp, Artificial Sequence
  rs2825583_F
gcttctttgtattgtcccaggactt

SEQ ID NO: 532 - DNA, 30 bp, Artificial Sequence
  rs2825583_R
aataattgcatgctctgtggtaatttcttt

SEQ ID NO: 533 - DNA, 23 bp, Artificial Sequence
  rs2825608_F
gtttacctgacactggaagcctt

SEQ ID NO: 534 - DNA, 29 bp, Artificial Sequence
  rs2825608_R
gccatacaattcacccacttaaaaagtac

SEQ ID NO: 535 - DNA, 29 bp, Artificial Sequence
  rs2825610_F
gttgatatagccttcttagctcaagactc

SEQ ID NO: 536 - DNA, 29 bp, Artificial Sequence
  rs2825610_R
aaatgtcatcctcactgattctcaagtac

SEQ ID NO: 537 - DNA, 23 bp, Artificial Sequence
  rs128365_F
tggcatgaagggaaaaatgcttc

SEQ ID NO: 538 - DNA, 24 bp, Artificial Sequence
  rs128365_R
ctagatgaaaacacccggtacaca

SEQ ID NO: 539 - DNA, 24 bp, Artificial Sequence
  rs2825688_F
tcatacagacgcgagtgaaattga

SEQ ID NO: 540 - DNA, 27 bp, Artificial Sequence
  rs2825688_R
ccacttccaaattttggaaaacgagag

SEQ ID NO: 541 - DNA, 25 bp, Artificial Sequence
  rs1474092_F
gggcaaatatatttccagtgcttca

SEQ ID NO: 542 - DNA, 30 bp, Artificial Sequence
  rs1474092_R
cgttgtggttaatattgtagcagcaaaaat

SEQ ID NO: 543 - DNA, 27 bp, Artificial Sequence
  rs2825824_F
agcaatgaaggacattctttctaggag

SEQ ID NO: 544 - DNA, 25 bp, Artificial Sequence
  rs2825824_R
ttccgtacagaaaatgcctaaacca

SEQ ID NO: 545 - DNA, 23 bp, Artificial Sequence
  rs377685_F
atcacctttaacacccttgcctt

SEQ ID NO: 546 - DNA, 28 bp, Artificial Sequence
  rs377685_R
cccaaggtagtctataattccagatgac

SEQ ID NO: 547 - DNA, 25 bp, Artificial Sequence
  rs12482714_F
tcagagagcatatgtaggtgtggat

SEQ ID NO: 548 - DNA, 26 bp, Artificial Sequence
  rs12482714_R
tgtgcatcctcaatatccatgatgag

SEQ ID NO: 549 - DNA, 25 bp, Artificial Sequence
  rs2826117_F
tgtttctatgaagcttgggcaatgt

SEQ ID NO: 550 - DNA, 27 bp, Artificial Sequence
  rs2826117_R
ggacatatttcagcccataacactaca

SEQ ID NO: 551 - DNA, 23 bp, Artificial Sequence
  rs977660_F
gaacaaggcgatcagttgaaact

SEQ ID NO: 552 - DNA, 27 bp, Artificial Sequence
  rs977660_R
tttccctgggatctcattgaagaattc

SEQ ID NO: 553 - DNA, 26 bp, Artificial Sequence
  rs1786427_F
caataatttctggtttgggcaggaaa

SEQ ID NO: 554 - DNA, 30 bp, Artificial Sequence
  rs1786427_R
ggctttttaatgctaaccactcaaaaatga

SEQ ID NO: 555 - DNA, 25 bp, Artificial Sequence
  rs2826259_F
cttgcttttacctgctgatatctgc

SEQ ID NO: 556 - DNA, 22 bp, Artificial Sequence
  rs2826259_R
ggatccctgagaaggtgtccaa

SEQ ID NO: 557 - DNA, 29 bp, Artificial Sequence
  rs1786388_F
gatcttaagtgttctcatcactcacacat

SEQ ID NO: 558 - DNA, 30 bp, Artificial Sequence
  rs1786388_R
tccaaccttaaacaacttcacaaaaatgtt

SEQ ID NO: 559 - DNA, 22 bp, Artificial Sequence
  rs8128523_F
ccttggtgtccttcccttgttg

SEQ ID NO: 560 - DNA, 24 bp, Artificial Sequence
  rs8128523_R
gatgctcctggctcatcttgttaa

SEQ ID NO: 561 - DNA, 27 bp, Artificial Sequence
  rs2826390_F
gctttgagaaaacgaaaaatgggaatg

SEQ ID NO: 562 - DNA, 30 bp, Artificial Sequence
  rs2826390_R
gctggaagtgtaagtaggaaaatagaatca

SEQ ID NO: 563 - DNA, 30 bp, Artificial Sequence
  rs2826392_F
ttaccagaagtttcaaaattctttgctgac

SEQ ID NO: 564 - DNA, 26 bp, Artificial Sequence
  rs2826392_R
gctctaaaagaccttttccaacatcc

SEQ ID NO: 565 - DNA, 24 bp, Artificial Sequence
  rs2826396_F
cagcatgctggaatccaattaacc

SEQ ID NO: 566 - DNA, 27 bp, Artificial Sequence
  rs2826396_R
cttttcctggttgattttcaaggttca

SEQ ID NO: 567 - DNA, 27 bp, Artificial Sequence
  rs2096905_F
tgtgtcaagtcacttggaataatagcc

SEQ ID NO: 568 - DNA, 28 bp, Artificial Sequence
  rs2096905_R
attagcagattatttggctctcaactgt

SEQ ID NO: 569 - DNA, 29 bp, Artificial Sequence
  rs13433508_F
ccatgtgtagaagagatttaatttgccca

SEQ ID NO: 570 - DNA, 23 bp, Artificial Sequence
  rs13433508_R
gctgctgctcccatgtcaaaata

SEQ ID NO: 571 - DNA, 25 bp, Artificial Sequence
  rs12483578_F
gtgattgtaacactgcactcaaacg

SEQ ID NO: 572 - DNA, 27 bp, Artificial Sequence
  rs12483578_R
gagtcttgagttcaaaaccttgactct

SEQ ID NO: 573 - DNA, 23 bp, Artificial Sequence
  rs2826399_F
gggacaaaacgacaacatttcct

SEQ ID NO: 574 - DNA, 29 bp, Artificial Sequence
  rs2826399_R
tccaccattaaaaatagtggcaaaaactg

SEQ ID NO: 575 - DNA, 27 bp, Artificial Sequence
  rs9983568_F
acttcaagacttctctcagctatgaga

SEQ ID NO: 576 - DNA, 26 bp, Artificial Sequence
  rs9983568_R
ctttccattaacagtgccctattacg

SEQ ID NO: 577 - DNA, 25 bp, Artificial Sequence
  rs8129332_F
agaattgacaaacgtaggagcagtt

SEQ ID NO: 578 - DNA, 29 bp, Artificial Sequence
  rs8129332_R
ttttccacaagtacaatggtaactgaaga

SEQ ID NO: 579 - DNA, 30 bp, Artificial Sequence
  rs229441_F
gaatgacaaatctgatcagatattgagggt

SEQ ID NO: 580 - DNA, 27 bp, Artificial Sequence
  rs229441_R
cctactttttggcattcaagtgcaaaa

SEQ ID NO: 581 - DNA, 23 bp, Artificial Sequence
  rs2826506_F
tggcagttgtatgacagctttca

SEQ ID NO: 582 - DNA, 30 bp, Artificial Sequence
  rs2826506_R
ccttctccagtcttcaactgaaatttaaca

SEQ ID NO: 583 - DNA, 23 bp, Artificial Sequence
  rs2014509_F
gccaaaatcaatgggctttttgc

SEQ ID NO: 584 - DNA, 25 bp, Artificial Sequence
  rs2014509_R
atctcctgtttacgcttcgatttga

SEQ ID NO: 585 - DNA, 30 bp, Artificial Sequence
  rs11096435_F
tcccagttttacttgtacaataattcctcg

SEQ ID NO: 586 - DNA, 30 bp, Artificial Sequence
  rs11096435_R
cccagacaatgtttgctttacatttattct

SEQ ID NO: 587 - DNA, 30 bp, Artificial Sequence
  rs13304202_F
cacacatcaatctacacaccatgtttttac

SEQ ID NO: 588 - DNA, 23 bp, Artificial Sequence
  rs13304202_R
tatccagggctgcctatcagaaa

SEQ ID NO: 589 - DNA, 29 bp, Artificial Sequence
  rs13304168_F
tcgctaatttattcacacaaagaaagtgc

SEQ ID NO: 590 - DNA, 25 bp, Artificial Sequence
  rs13304168_R
cagaaacatagtctggatggcagaa

SEQ ID NO: 591 - DNA, 22 bp, Artificial Sequence
  rs16980558_F
tgcgcctcaccaaaagactaat

SEQ ID NO: 592 - DNA, 30 bp, Artificial Sequence
  rs16980558_R
gctcctggcgtgtatttttaaatttcttaa

SEQ ID NO: 593 - DNA, 22 bp, Artificial Sequence
  rs16980586_F
aaggtacaggcacgtctagaga

SEQ ID NO: 594 - DNA, 30 bp, Artificial Sequence
  rs16980586_R
ccctacactttgttggaaaatagttgattt

SEQ ID NO: 595 - DNA, 27 bp, Artificial Sequence
  rs16980588_F
tgtcaattaaacacagtccacattcca

SEQ ID NO: 596 - DNA, 28 bp, Artificial Sequence
  rs16980588_R
tttagaggaatcagtggtttgtttgagt

SEQ ID NO: 597 - DNA, 22 bp, Artificial Sequence
  rs9786824_F
gaagctggttgaagtctggaca

SEQ ID NO: 598 - DNA, 29 bp, Artificial Sequence
  rs9786824_R
acaatcagaaaatcactcaattagcagga

SEQ ID NO: 599 - DNA, 29 bp, Artificial Sequence
  rs765557_F
ccttttatttttctagcaaactgtgggat

SEQ ID NO: 600 - DNA, 30 bp, Artificial Sequence
  rs765557_R
gctgatatgtcataatacggaagagtaagt

SEQ ID NO: 601 - DNA, 29 bp, Artificial Sequence
  rs9786386_F
gaatatatcttgacccatgcatgttttgg

SEQ ID NO: 602 - DNA, 30 bp, Artificial Sequence
  rs9786386_R
ggtgaagtgaaatagcagctaccatttata

SEQ ID NO: 603 - DNA, 30 bp, Artificial Sequence
  rs17307670_F
cttccagtaaatttcagctttctgttttga

SEQ ID NO: 604 - DNA, 24 bp, Artificial Sequence
  rs17307670_R
gctcacatttggcagggatactag

SEQ ID NO: 605 - DNA, 22 bp, Artificial Sequence
  rs9786876_F
tctgagacctagccacatggat

SEQ ID NO: 606 - DNA, 30 bp, Artificial Sequence
  rs9786876_R
ggcaagagactagtgaagatataaatgtga

SEQ ID NO: 607 - DNA, 25 bp, Artificial Sequence
  rs9785659_F
catgttgggcataattgtctcgatg

SEQ ID NO: 608 - DNA, 27 bp, Artificial Sequence
  rs9785659_R
gggttgttgatccatatggagttatga

SEQ ID NO: 609 - DNA, 22 bp, Artificial Sequence
  rs9786121_F
ggtagcaagccacctagtttca

SEQ ID NO: 610 - DNA, 24 bp, Artificial Sequence
  rs9786121_R
ccactagaccatggtccacttaga

SEQ ID NO: 611 - DNA, 25 bp, Artificial Sequence
  rs9786111_F
ccaattgaacaaactcacatcgaca

SEQ ID NO: 612 - DNA, 30 bp, Artificial Sequence
  rs9786111_R
catgatatagatcatttgccaccacataca

SEQ ID NO: 613 - DNA, 28 bp, Artificial Sequence
  rs9786276_F
caccataagtacatgagaaatgcaagac

SEQ ID NO: 614 - DNA, 25 bp, Artificial Sequence
  rs9786276_R
cattgagacccagtgagttatgtct

SEQ ID NO: 615 - DNA, 22 bp, Artificial Sequence
  rs2161775_F
gattggaggacagcaagtccaa

SEQ ID NO: 616 - DNA, 24 bp, Artificial Sequence
  rs2161775_R
gaatttgcactgggtaagtgtctc

SEQ ID NO: 617 - DNA, 27 bp, Artificial Sequence
  rs9786101_F
ggaagaagcatggctgatcaattaatg

SEQ ID NO: 618 - DNA, 23 bp, Artificial Sequence
  rs9786101_R
tgtacccagtaactcaccgtttg

SEQ ID NO: 619 - DNA, 24 bp, Artificial Sequence
  rs3991109_F
ccctcagcctcccaatatatgaag

SEQ ID NO: 620 - DNA, 30 bp, Artificial Sequence
  rs3991109_R
agatgtcagagctcaattctagactatgaa

SEQ ID NO: 621 - DNA, 23 bp, Artificial Sequence
  rs9785716_F
gggaacccacttctctgaagaat

SEQ ID NO: 622 - DNA, 25 bp, Artificial Sequence
  rs9785716_R
ggctctatgatcagcaggtgattat

SEQ ID NO: 623 - DNA, 25 bp, Artificial Sequence
  rs7067226_F
accagatctacctcacaagagatgt

SEQ ID NO: 624 - DNA, 26 bp, Artificial Sequence
  rs7067226_R
tgcttggtagatttccttccatttcc

SEQ ID NO: 625 - DNA, 24 bp, Artificial Sequence
  rs7067297_F
ccaggatgggaatgagagtaacca

SEQ ID NO: 626 - DNA, 29 bp, Artificial Sequence
  rs7067297_R
gaataaagaatgcctactctataggccaa

SEQ ID NO: 627 - DNA, 27 bp, Artificial Sequence
  rs9785897_F
ctttagaatgttgagctttttgcccat

SEQ ID NO: 628 - DNA, 30 bp, Artificial Sequence
  rs9785897_R
tgtcattcaactgaagatgacaagatagag

SEQ ID NO: 629 - DNA, 22 bp, Artificial Sequence
  rs1276034_F
gtgacaattgcaagcctggaat

SEQ ID NO: 630 - DNA, 28 bp, Artificial Sequence
  rs1276034_R
gactctgtttcacttttatttgcatggt

SEQ ID NO: 631 - DNA, 27 bp, Artificial Sequence
  rs2058276_F
gatcctaagactaagagcaaaaagcca

SEQ ID NO: 632 - DNA, 30 bp, Artificial Sequence
  rs2058276_R
acttttcctaagacatcagaacaagacttt

SEQ ID NO: 633 - DNA, 26 bp, Artificial Sequence
  rs1358368_F
ccagacaagagtgctaaaaaccaagt

SEQ ID NO: 634 - DNA, 25 bp, Artificial Sequence
  rs1358368_R
ctatctccacggttgcatctatctg

SEQ ID NO: 635 - DNA, 28 bp, Artificial Sequence
  rs9786773_F
caacacacagtatgcccatgtaataaac

SEQ ID NO: 636 - DNA, 27 bp, Artificial Sequence
  rs9786773_R
cttcatagcgtttatgaggttccaaac

SEQ ID NO: 637 - DNA, 23 bp, Artificial Sequence
  rs9786885_F
caggcagagtctggagtgattaa

SEQ ID NO: 638 - DNA, 33 bp, Artificial Sequence
  rs9786885_R
tcaaaatgacttttatttccctaggatgtttca

SEQ ID NO: 639 - DNA, 31 bp, Artificial Sequence
  rs2571764_F
tatagagatttcccgtatcttctctacccta

SEQ ID NO: 640 - DNA, 33 bp, Artificial Sequence
  rs2571764_R
tggaaatggctacatactgtatgattctaacta

SEQ ID NO: 641 - DNA, 26 bp, Artificial Sequence
  rs3096835_F
ttttaaaattggccaggcttgatagc

SEQ ID NO: 642 - DNA, 29 bp, Artificial Sequence
  rs3096835_R
ctcgttttaccataatatccaagtctggt

SEQ ID NO: 643 - DNA, 29 bp, Artificial Sequence
  rs1864469_F
ctcatgtttctatagcaagtaatgagggt

SEQ ID NO: 644 - DNA, 25 bp, Artificial Sequence
  rs1864469_R
gcacttcatgcaaatgtcatgactc

SEQ ID NO: 645 - DNA, 29 bp, Artificial Sequence
  rs9786291_F
tcctgaatgtaacaaggaaataaagcaca

SEQ ID NO: 646 - DNA, 26 bp, Artificial Sequence
  rs9786291_R
agaattgcttgaagtcatagtgcaga

SEQ ID NO: 647 - DNA, 27 bp, Artificial Sequence
  rs9786140_F
ttgcagacaacccaaagagagacacta

SEQ ID NO: 648 - DNA, 23 bp, Artificial Sequence
  rs9786140_R
gataggatgccacaagtttgcac

SEQ ID NO: 649 - DNA, 30 bp, Artificial Sequence
  rs11096453_F
tacatgttcagatttgggagataattctgg

SEQ ID NO: 650 - DNA, 22 bp, Artificial Sequence
  rs11096453_R
ccatgtcccatggtcaagtctc

SEQ ID NO: 651 - DNA, 28 bp, Artificial Sequence
  rs9786194_F
atgttatgaaatagcagctgctttttcc

SEQ ID NO: 652 - DNA, 30 bp, Artificial Sequence
  rs9786194_R
ccacagtcgttttgtcatctgttatattct

SEQ ID NO: 653 - DNA, 120 bp, Artificial Sequence
  Marker sequence_rs149098
  misc_feature (81)..(81): n is a, c, g, or t
taagtatattctttaaggctattttctccacctcctcttctcacattttaaattatacccaggcaatttcttctatgcacnttggttctctttctttaccctagctctttctcatgatct

SEQ ID NO: 654 - DNA, 21 bp, Artificial Sequence
  rs149098 forward primer
tctccacctcctcttctcaca

SEQ ID NO: 655 - DNA, 24 bp, Artificial Sequence
  rs149098 reverse primer
gctagggtaaagaaagagaaccaa